(19) United States
(12) Patent Application Publication
(10) Pub. No.: US 2005/0048502 A1
(43) Pub. Date: Mar. 3, 2005

(54) Human SACH-related gene variants associated with cancers

(76) Inventor: Ken-Shwo Dai, Hsinchu (TW)

Correspondence Address: Ladas & Parry, 26 West 61st Street, New York, NY, 10023, US

(21) Appl. No.: 10/653,680

(22) Filed: Sep. 2, 2003

(51) Int. Cl. C12Q 1/68; G01N 33/574; C07H 21/04; C07K 14/705

(52) U.S. Cl. 435/6; 435/7.23; 435/320.1; 435/325; 435/69.1; 530/350; 536/23.2

(57) ABSTRACT

The invention relates to the nucleic acid sequences of three novel human SACH-related gene variants (SACHV1, SACHV2 and SACHV3) and the polypeptides encoded by SACHV1, SACHV2 and SACHV3. The invention also relates to the process for producing the polypeptides encoded by SACHV1, SACHV2 and SACHV3. The invention further relates to the use of the nucleic acid of SACHV1, SACHV2 and SACHV3, and the polypeptide encoded by SACHV1, SACHV2 and SACHV3 in diagnosing diseases associated with the deficiency of human SACHV genes, in particular SCLC and lung carcinoids.

### FIELD OF THE INVENTION

The invention relates to the nucleic acid sequences of three novel human SACH-related gene variants (SACHV1, SACHV2 and SACHV3) and the polypeptides encoded thereby, the preparation process thereof, and the uses of the same in diagnosing diseases associated with the deficiency of the gene variants, in particular, human cancers, e.g., small cell lung cancer or lung carcinoids.

### BACKGROUND OF THE INVENTION

Lung cancer is one of the major causes of cancer-related deaths in the world. There are two primary types of lung cancers: small cell lung cancer (SCLC) and non-small cell lung cancer (NSCLC) (Carney, (1992a) Curr. Opin. Oncol. 4:292-8). Small cell lung cancer accounts for approximately 25% of lung cancer and spreads aggressively (Smyth et al. (1986) Q J. Med. 61: 969-76; Carney, (1992b) Lancet 339: 843-6). Non-small cell lung cancer represents the majority (about 75%) of lung cancer, and is further divided into three main subtypes: squamous cell carcinoma, adenocarcinoma, and large cell carcinoma (Ihde and Minutesna, (1991) Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggests that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes associated with the regulation of transcription. Many helix-loop-helix (HLH) proteins, a family of transcription factors, have been reported to play a role in the regulation of developmental processes (Massari and Murre, (2000) Mol Cell Biol. 20:429-40). Amoung these HLH proteins, human achaete-scute homology 1 was found to be expressed highly in SCLC (Ball et al. (1993) Proc Natl Acad Sci USA. 90:5648-52; Bhattacharjee et al. (2001) Proc Natl Acad Sci USA. 98:13790-5; Sugita et al. (2002) Cancer Res. 62:3971-9). Decreased expression of the human achaete-scute homology 1 has been found to be associated with the cell cycle arrest in SCLC cells (Sriuranpong et al. (2001) Cancer Res. 61:3200-5). Recently, a novel gene (GenBank accession # BC001638) isolated from SCLC shows that its sequence is similar to the human achaete-scute homology 1. These results suggest that the gene variants of this novel gene (we named it SACH for the purpose of the present study) may be important targets for diagnostic markers of cancers.

### SUMMARY OF THE INVENTION

The invention provides three SACH-related gene variants, SACHV1, SACHV2 and SACHV3, found in human SCLC and lung carcinoid, and the polypeptide sequences encoded thereby, which are useful in the diagnosis of the diseases associated with the deficiency of human SACH gene, in particular, cancers, preferably SCLC and lung carcinoid.

The invention further provides expression vectors and host cells for expressing SACHV1, SACHV2 and SACHV3.

The invention further provides a method for producing the polypeptides encoded by SACHV1, SACHV2 and SACHV3.

The invention further provides antibodies specifically binding to the polypeptides encoded by SACHV1, SACHV2 and SACHV3.

The invention also provides methods for diagnosing the diseases associated with the deficiency of human SACH gene, in particular, cancers, preferable SCLC and lung carcinoid.

### BRIEF DESCRIPTION OF THE DRAWINGS

FIG. 1A to 1E show the nucleic acid sequence of SACHV1 (SEQ ID NO: 1) and the amino acid sequence encoded thereby (SEQ ID NO: 2).

FIGS. 2A to 2E show the nucleic acid sequence of SACHV2 (SEQ ID NO: 3) and the amino acid sequence encoded thereby (SEQ ID NO: 4).

FIGS. 3A to 3E show the nucleic acid sequence of SACHV3 (SEQ ID NO: 5) and the amino acid sequence encoded thereby (SEQ ID NO: 6).

FIGS. 4A to 4U show the nucleotide sequence alignment between human SACH gene and SACHV1, SACHV2 and SACHV3.

FIGS. 5A to 5B show the amino acid sequence alignment among human SACH and the polypeptides encoded by SACHV1, SACHV2 and SACHV3.

### DETAILED DESCRIPTION OF THE INVENTION

According to the invention, all technical and scientific terms used have the same meanings as commonly understood by persons skilled in the art.

The term “antibody,” as used herein, denotes intact molecules (a polypeptide or group of polypeptides) as well as fragments thereof, such as Fab, R(ab′)₂, and Fv fragments, which are capable of binding the epitopic determinutesant. Antibodies are produced by specialized B cells after stimulation by an antigen. Structurally, an antibody consists of four subunits including two heavy chains and two light chains. The internal surface shape and charge distribution of the antibody binding domain are complementary to the features of an antigen. Thus, an antibody can specifically act against the antigen in an immune response.

The term “base pair (bp),” as used herein, denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402),” as used herein, denotes programs for evaluation of homologies between a query sequence (amino or nucleic acid) and a test sequence as described by Altschul et al. (Nucleic Acids Res. 25: 3389-3402, 1997). Specific BLAST programs are described as follows:
(1) BLASTN compares a nucleotide query sequence against a nucleotide sequence database;
(2) BLASTP compares an amino acid query sequence against a protein sequence database;
(3) BLASTX compares the six-frame conceptual translation products of a query nucleotide sequence against a protein sequence database;
(4) TBLASTN compares a query protein sequence against a nucleotide sequence database translated in all six reading frames; and
(5) TBLASTX compares the six-frame translations of a nucleotide query sequence against the six-frame translations of a nucleotide sequence database.

The term “cDNA,” as used herein, denotes nucleic acids that are synthesized from a mRNA template using reverse transcriptase.

The term “cDNA library,” as used herein, denotes a library composed of complementary DNAs which are reverse-transcribed from mRNAs.

The term “complement,” as used herein, denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion,” as used herein, denotes a removal of a portion of one or more amino acid residues/nucleotides from a gene.

The term “expressed sequence tags (ESTs),” as used herein, denotes a short (200 to 500 base pairs) nucleotide sequence that derives from either 5′ or 3′ end of a cDNA.

The term “expression vector,” as used herein, denotes nucleic acid constructs which contain a cloning site for introducing the DNA into a vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell,” as used herein, denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition,” as used herein, denotes the addition of a portion of one or more amino acid residues/nucleotides to a gene.

The term “in silico,” as used herein, denotes a process of using computational methods (e.g., BLAST) to analyze DNA sequences.

The term “polymerase chain reaction (PCR),” as used herein, denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “primer,” as used herein, denotes a single-stranded synthetic oligonucleotide designed to hybridize to a particular template DNA sequence. The forward primer is the one complementary to one strand at the 5′-end of the DNA sequence. The reverse primer is the one complementary to the other strand at the 3′-end of the DNA sequence.

The term “protein” or “polypeptide,” as used herein, denotes a sequence of amino acids in a specific order that can be encoded by a gene or by a recombinant DNA. It can also be chemically synthesized.

The term “nucleic acid sequence” or “polynucleotide,” as used herein, denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR),” as used herein, denotes a process which transcribes mRNA to a complementary DNA strand using reverse transcriptase followed by polymerase chain reaction to amplify the specific fragment of DNA sequences.

The term “transformation,” as used herein, denotes a process describing the uptake, incorporation, and expression of exogenous DNA by prokaryotic host cells.

The term “transfection,” as used herein, is a process describing the uptake, incorporation, and expression of exogenous DNA by eukaryotic host cells.

The term “variant,” as used herein, denotes a fragment of sequence (nucleotide or amino acid) inserted or deleted by one or more nucleotides/amino acids.

In the first aspect, the subject invention provides the nucleotide sequences of SACHV1, SACHV2 and SACHV3, and the polypeptides encoded by the three novel human SACH-related gene variants and fragments thereof.

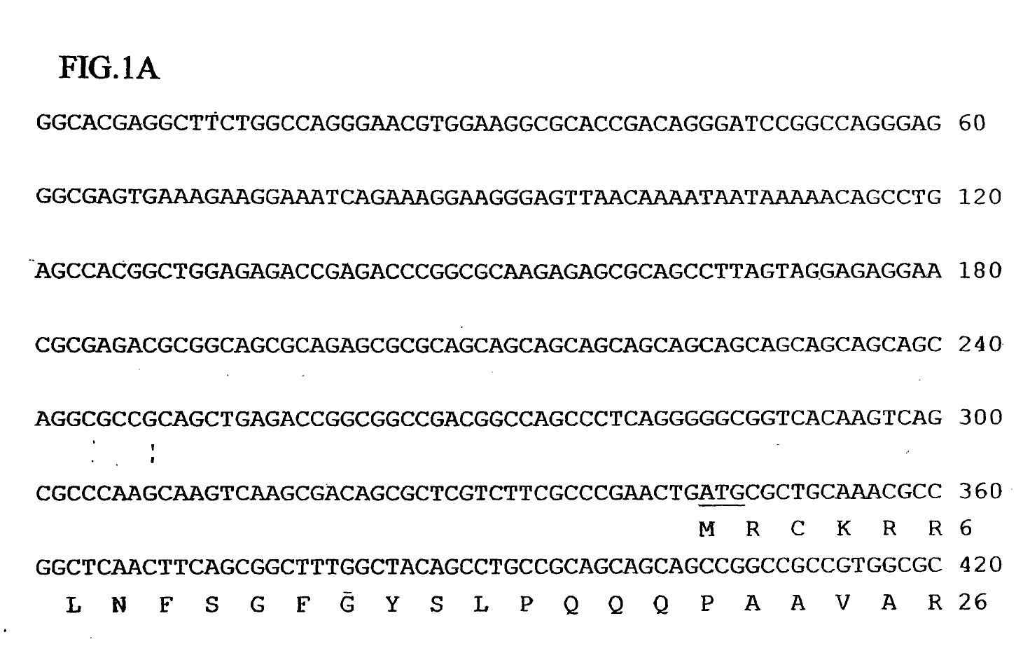

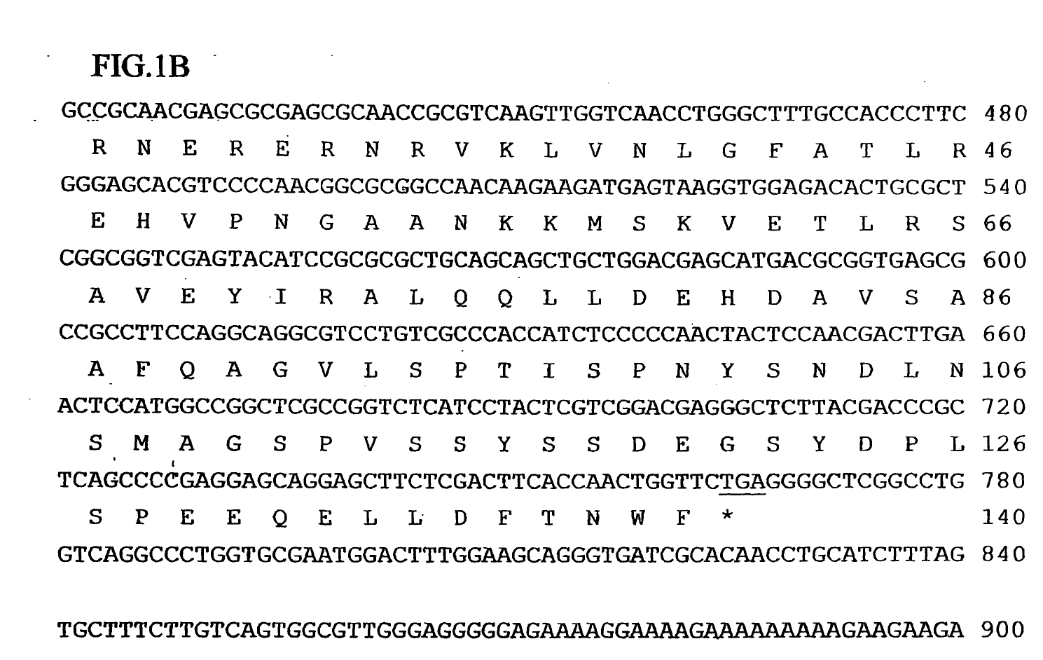

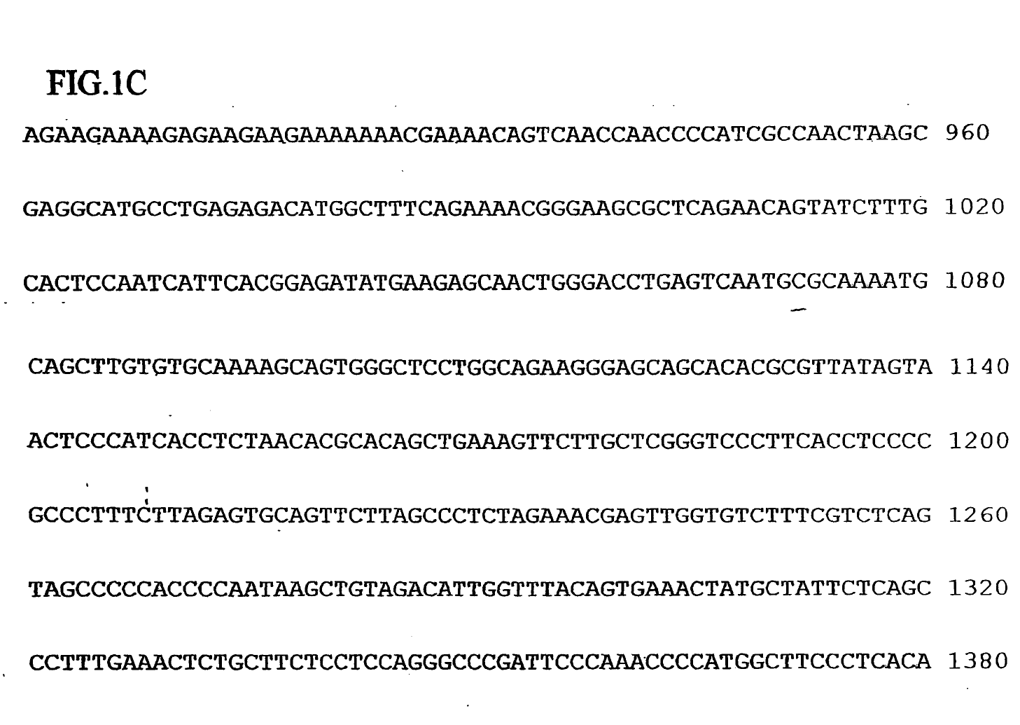

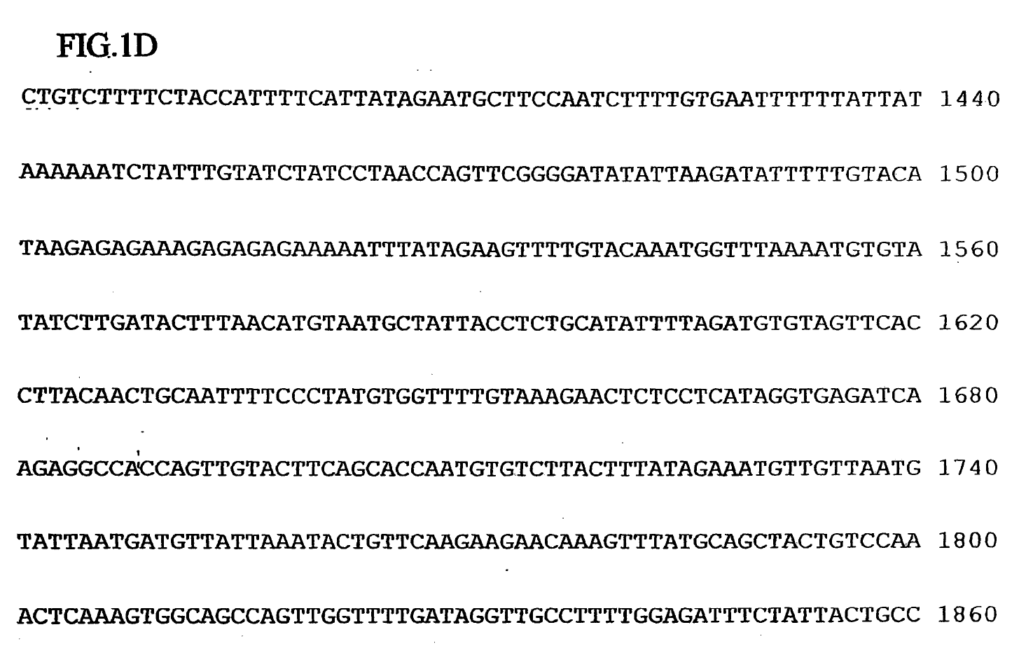

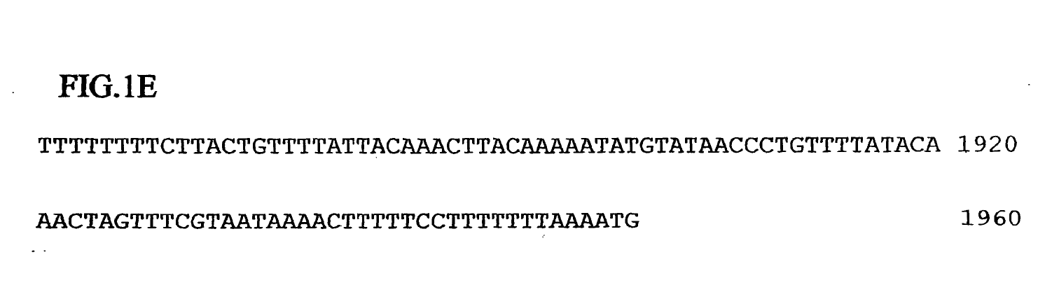

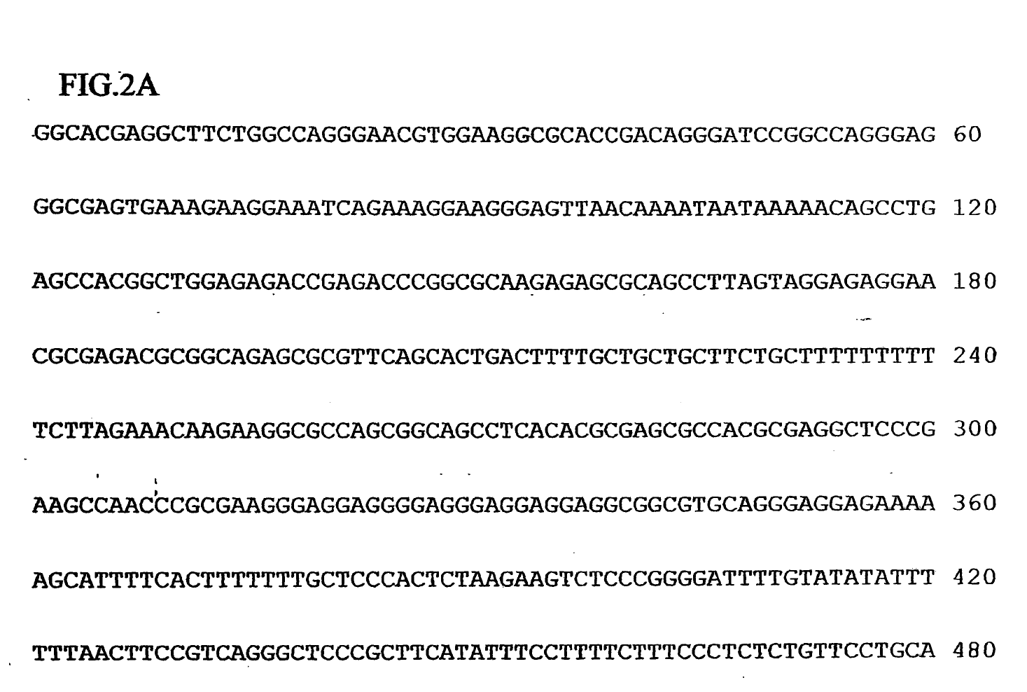

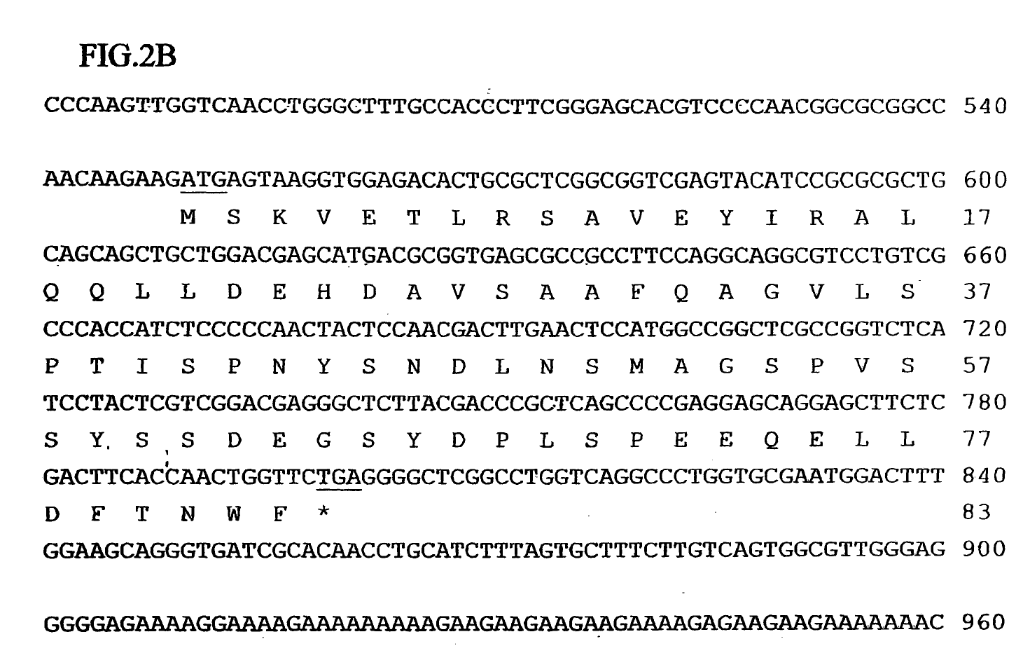

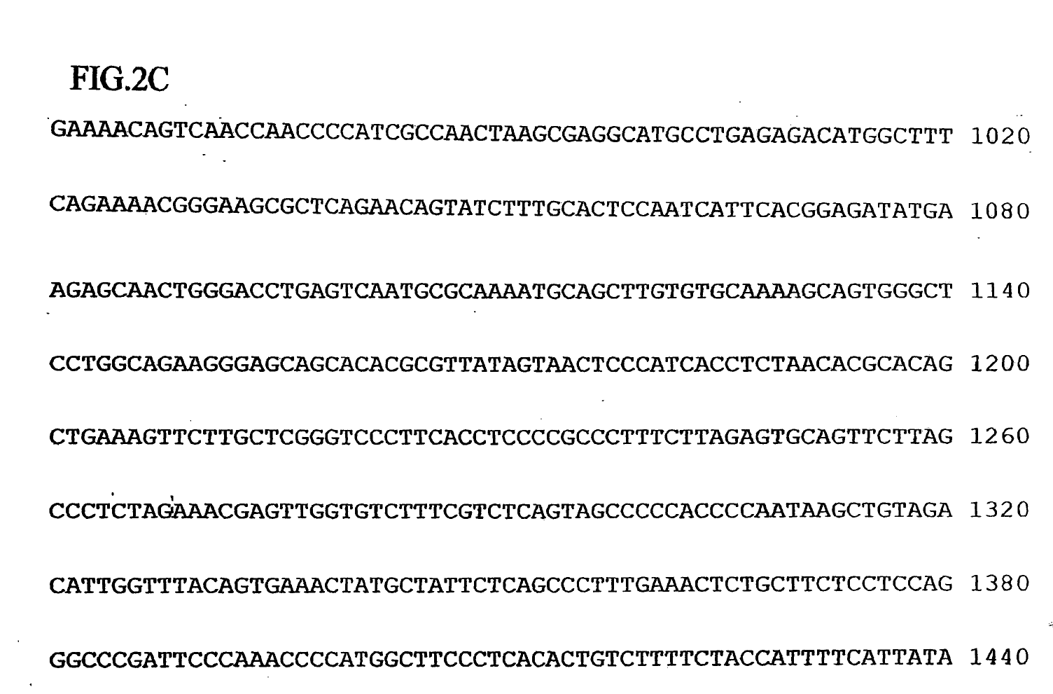

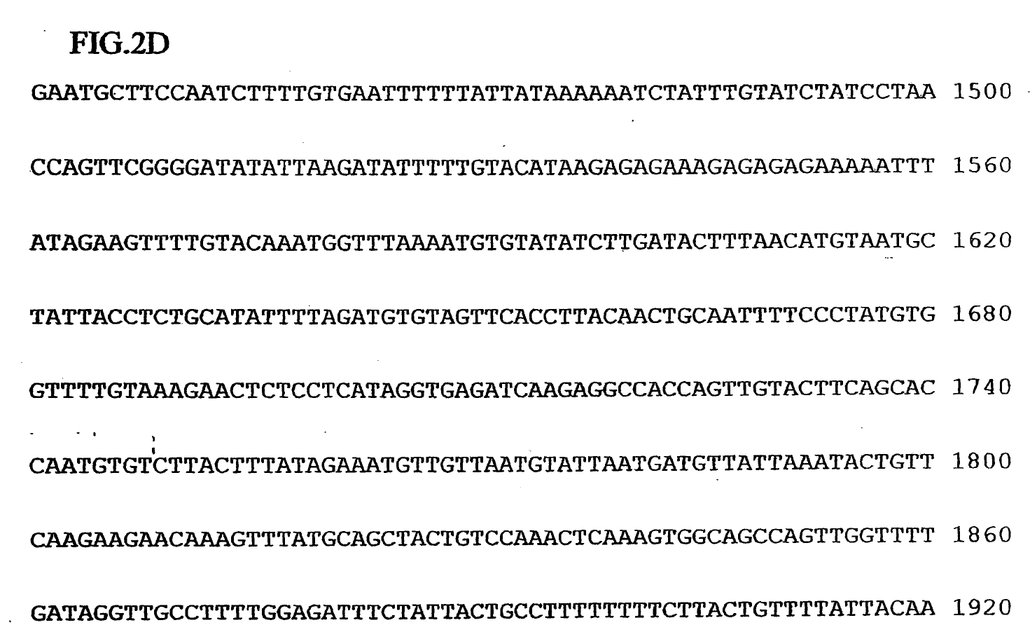

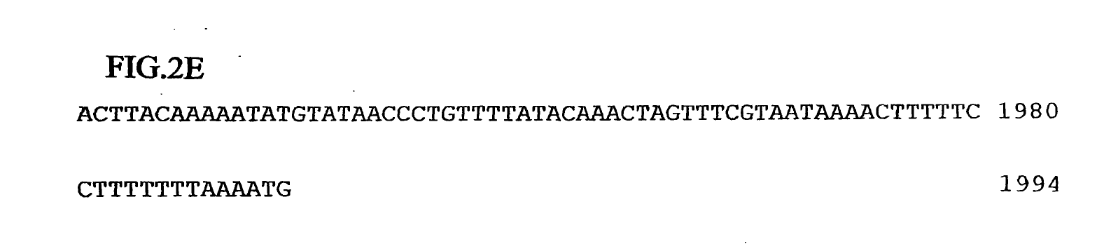

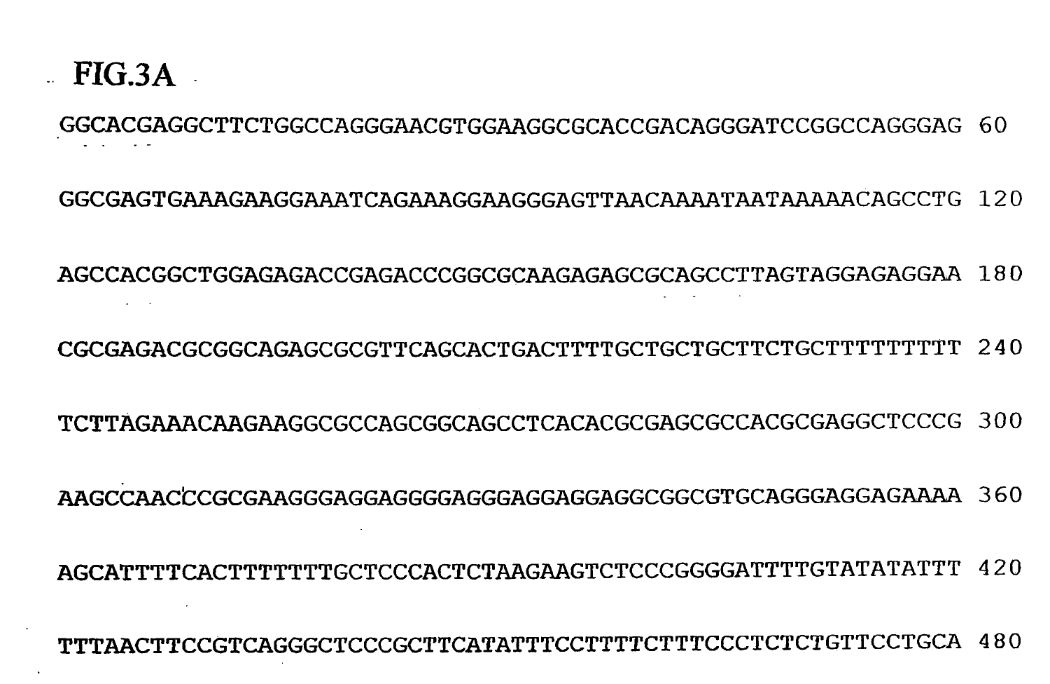

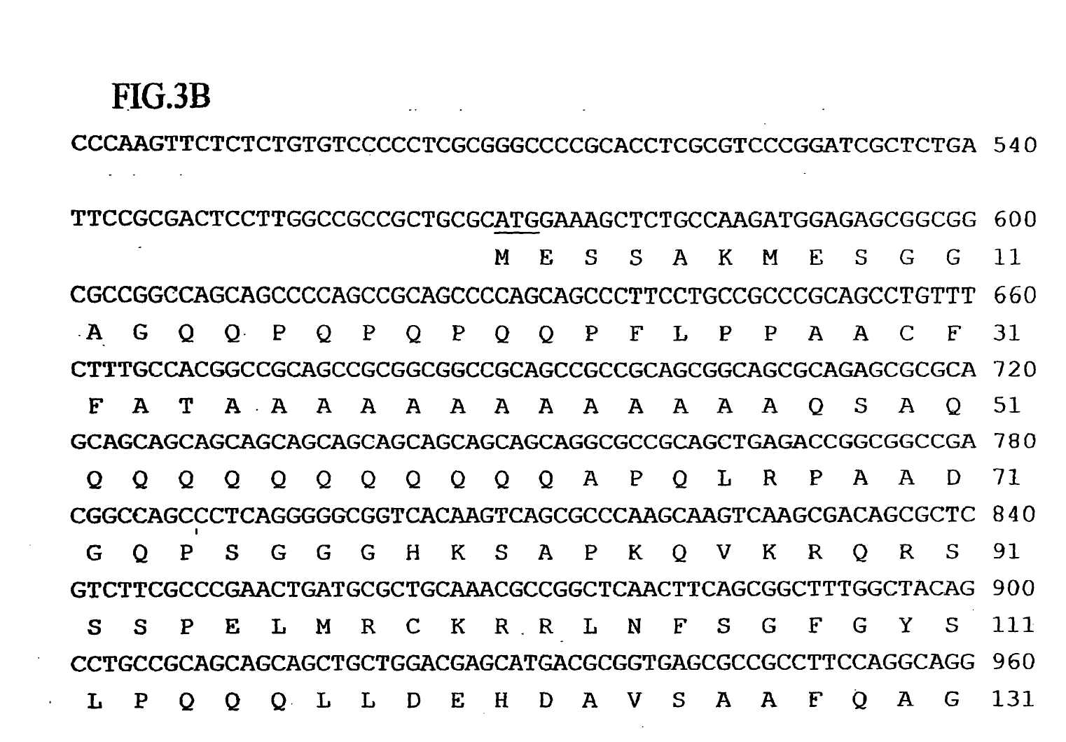

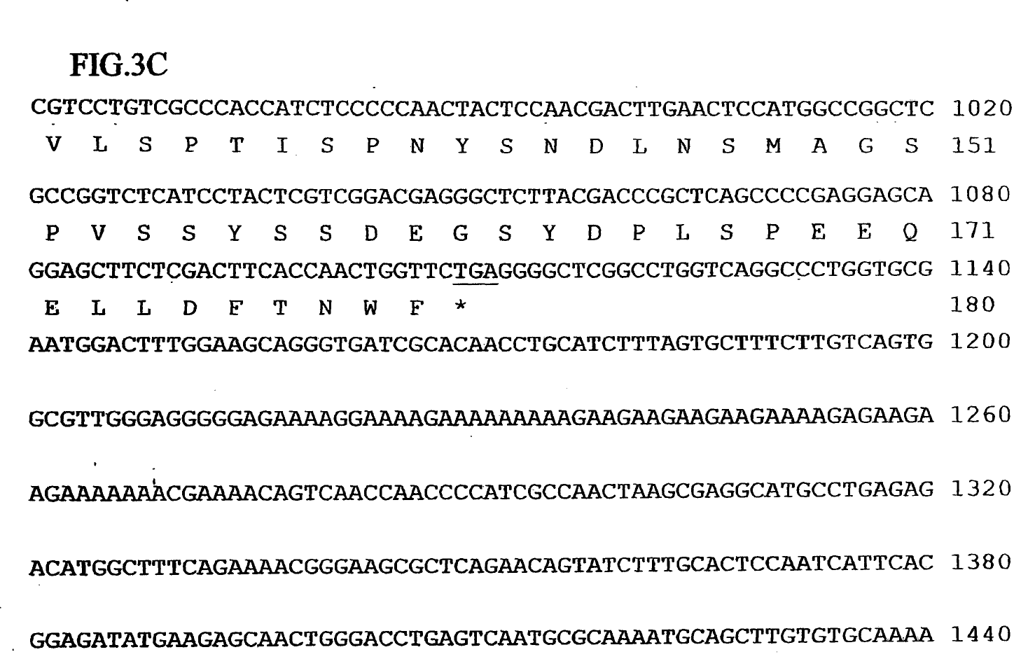

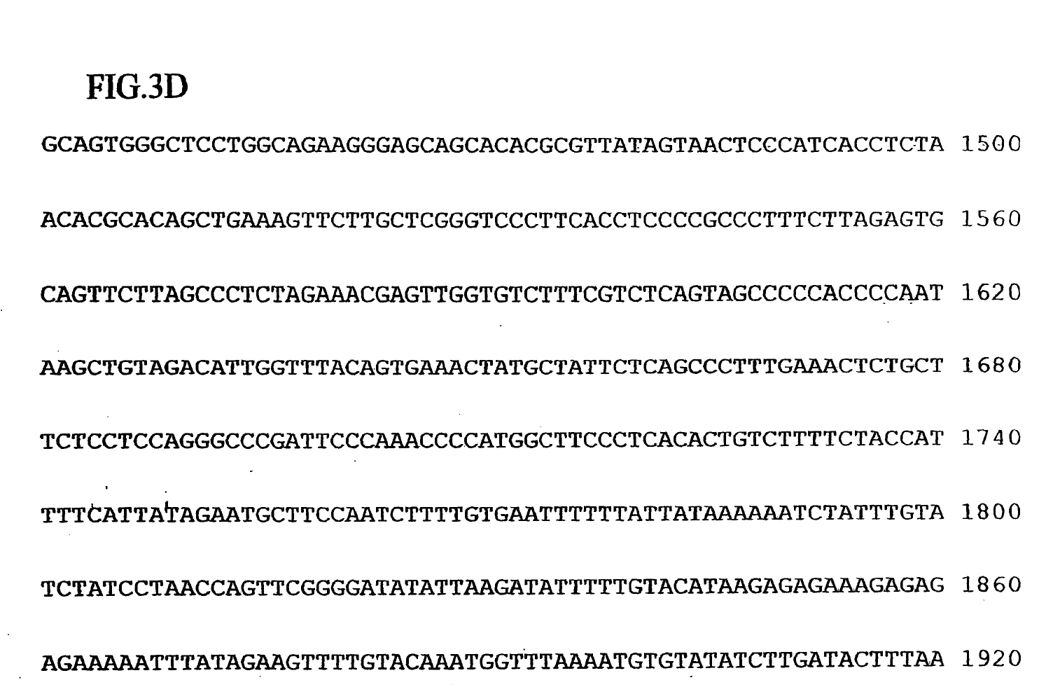

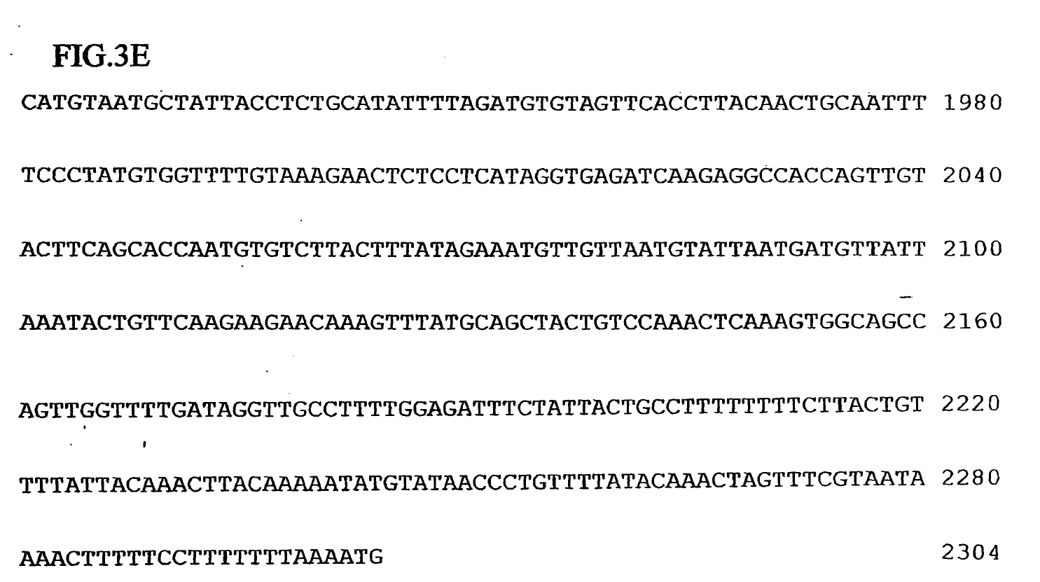

According to the invention, human SACH cDNA sequence was used to query a human SCLC and a lung carcinoid EST database using BLAST program to search for SACH-related gene variants. Three human cDNA partial sequences (i.e., ESTs) deposited in the databases showing similar to SACH were isolated and sequenced. These clones (named SACHV1, SACHV2 and SACHV3) were isolated. FIGS. 1, 2 and 3 show the nucleic acid sequences (SEQ ID NOs: 1, 3 and 5) of the variants (SACHV1, SACHV2 and SACHV3) and the corresponding amino acid sequences (SEQ ID NOs: 2, 4 and 6) encoded thereby.

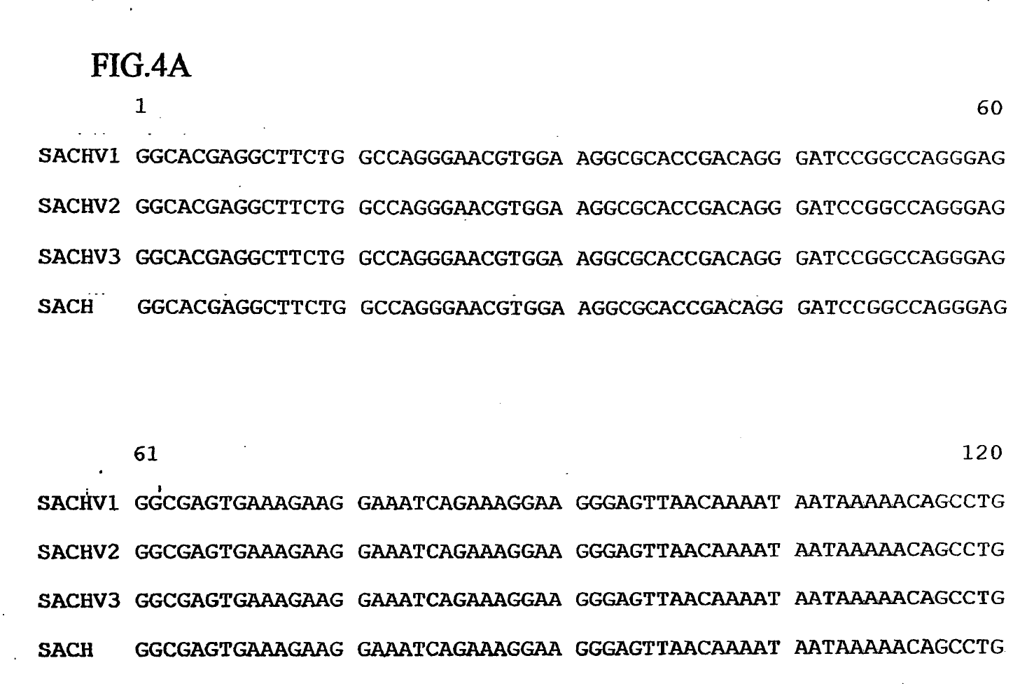

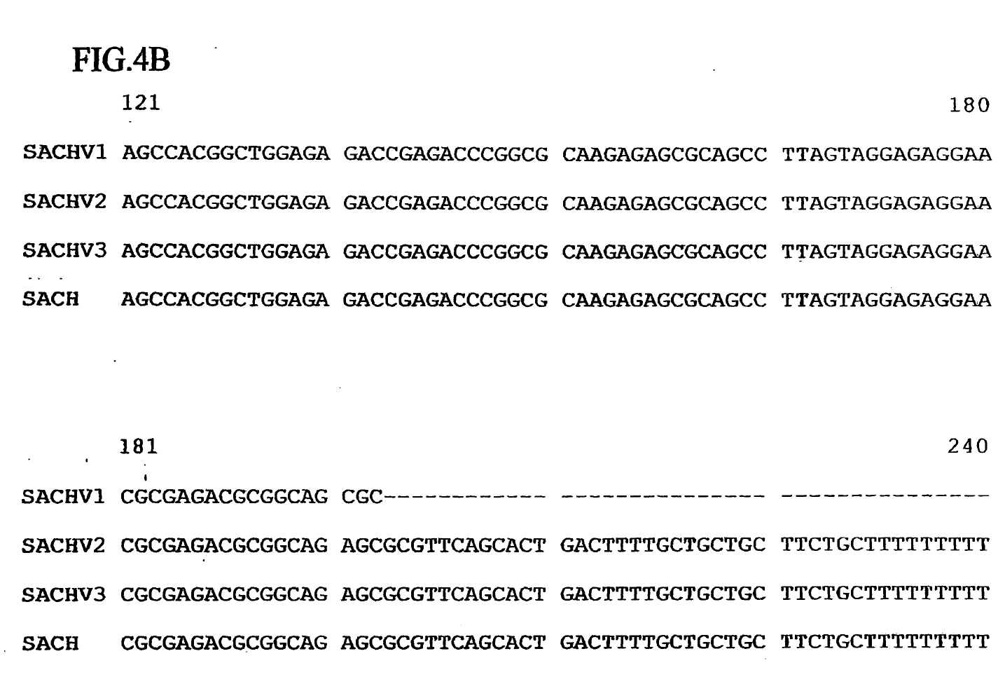

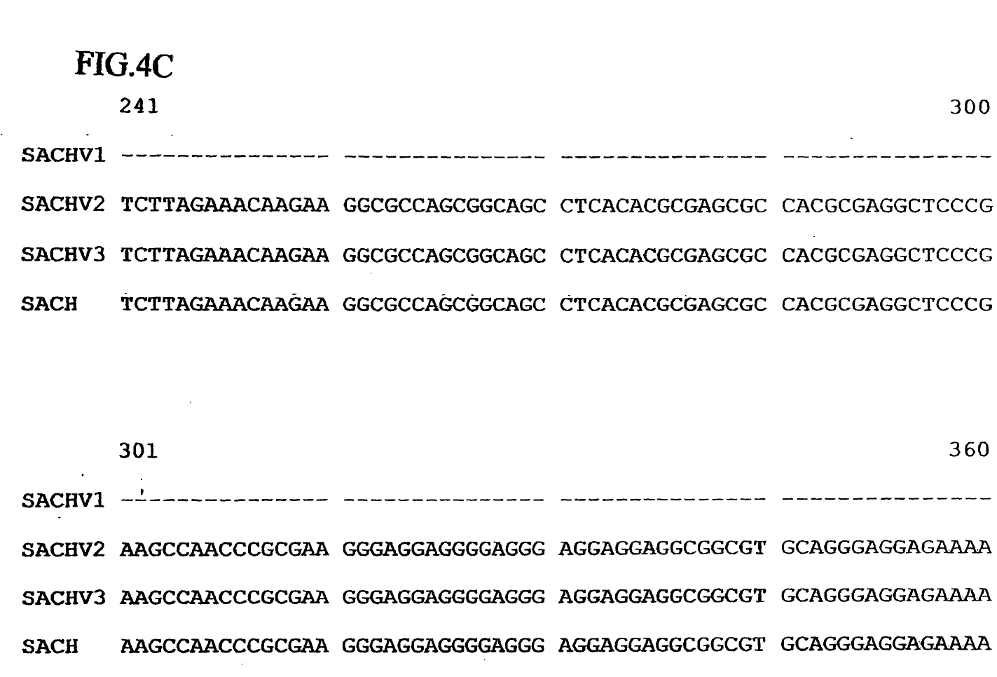

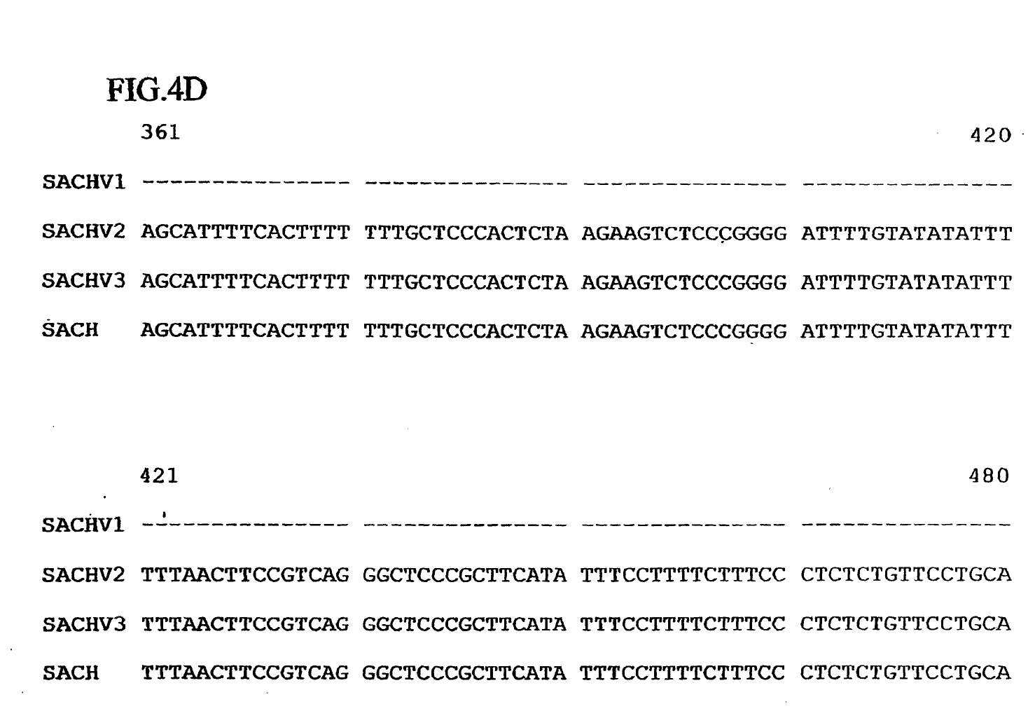

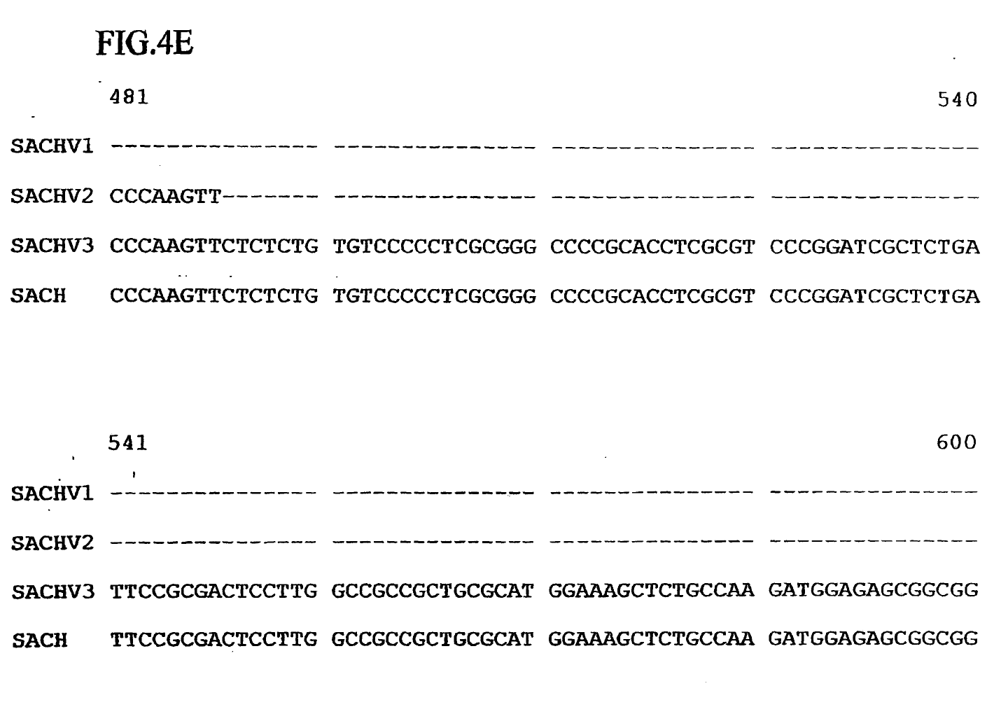

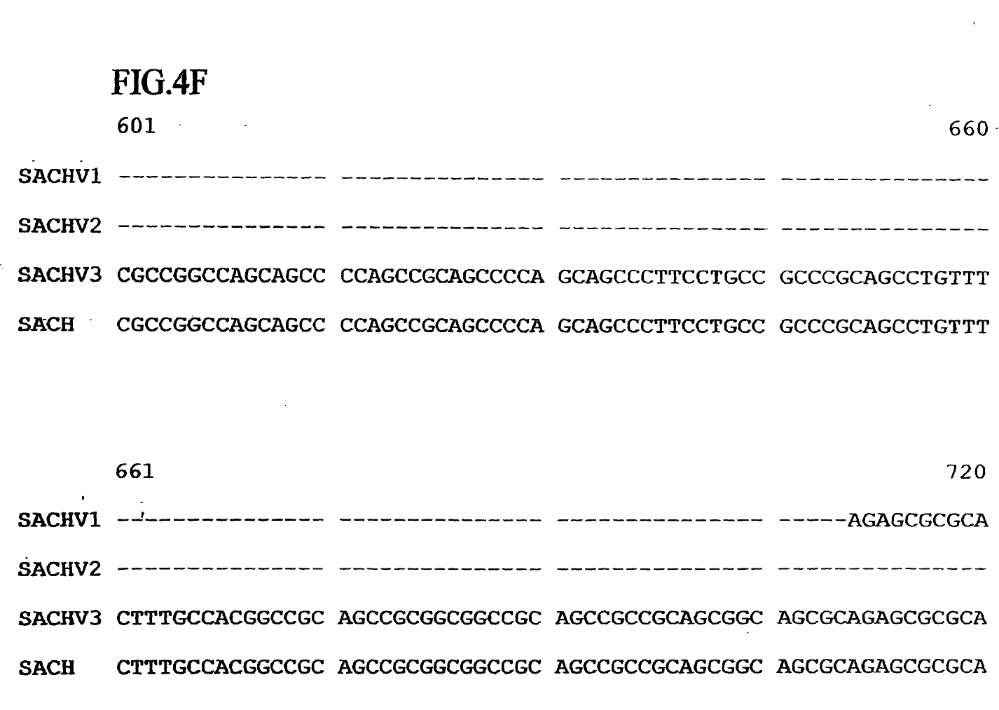

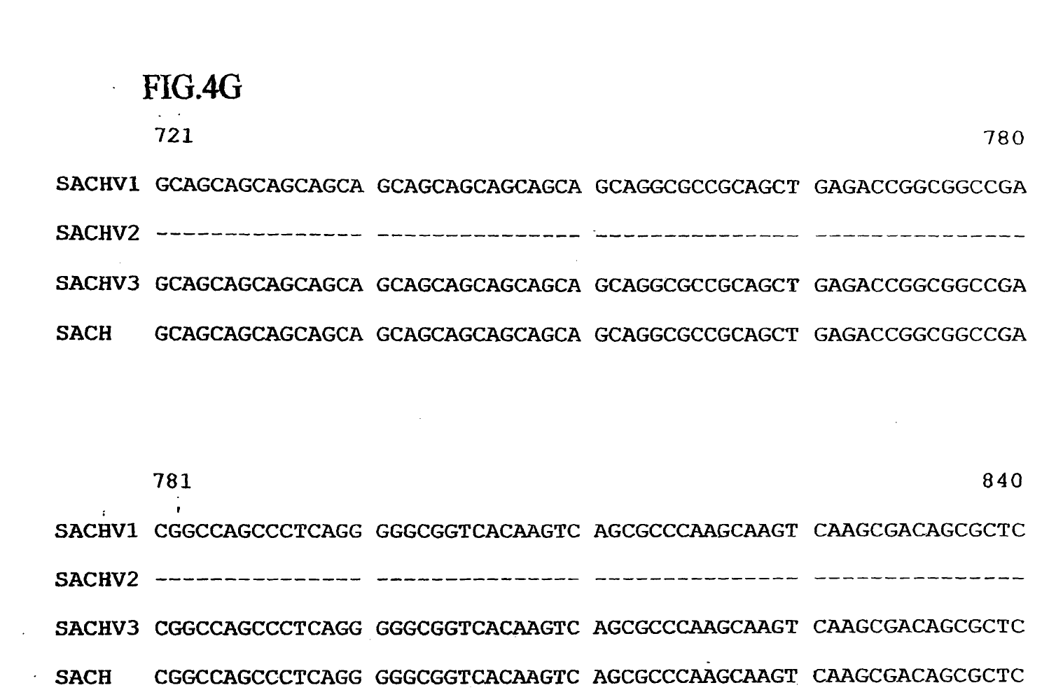

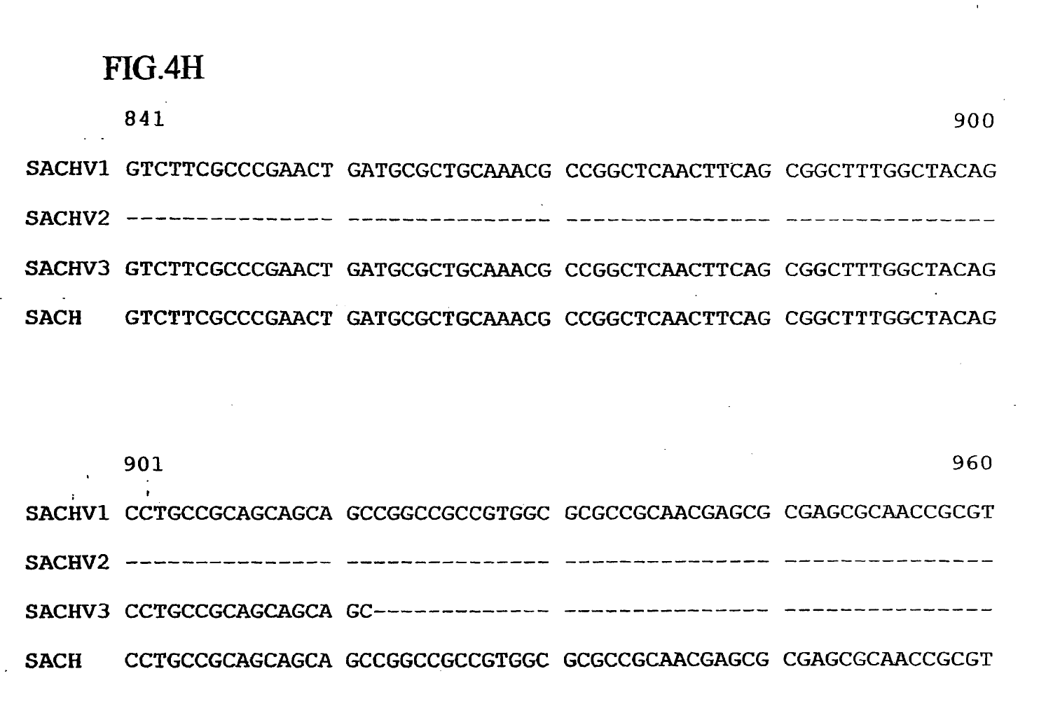

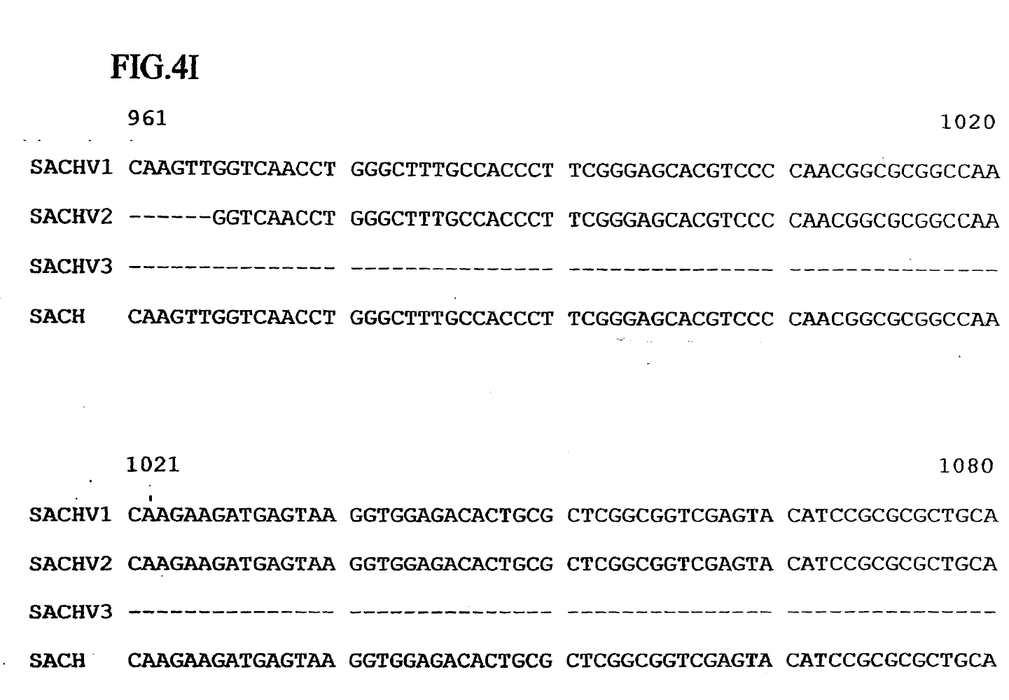

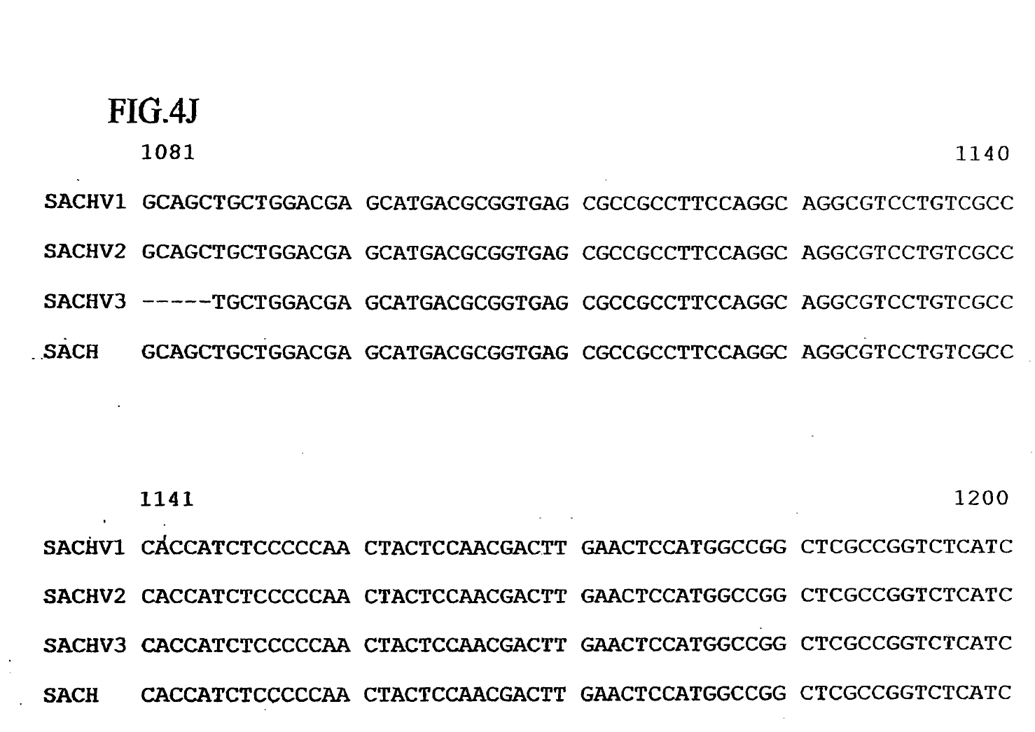

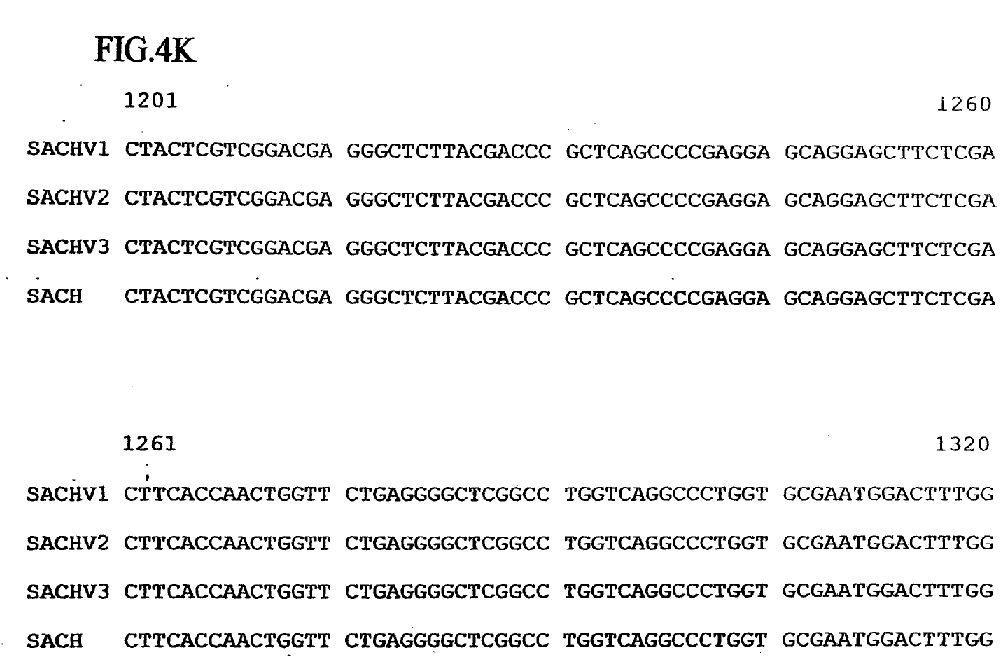

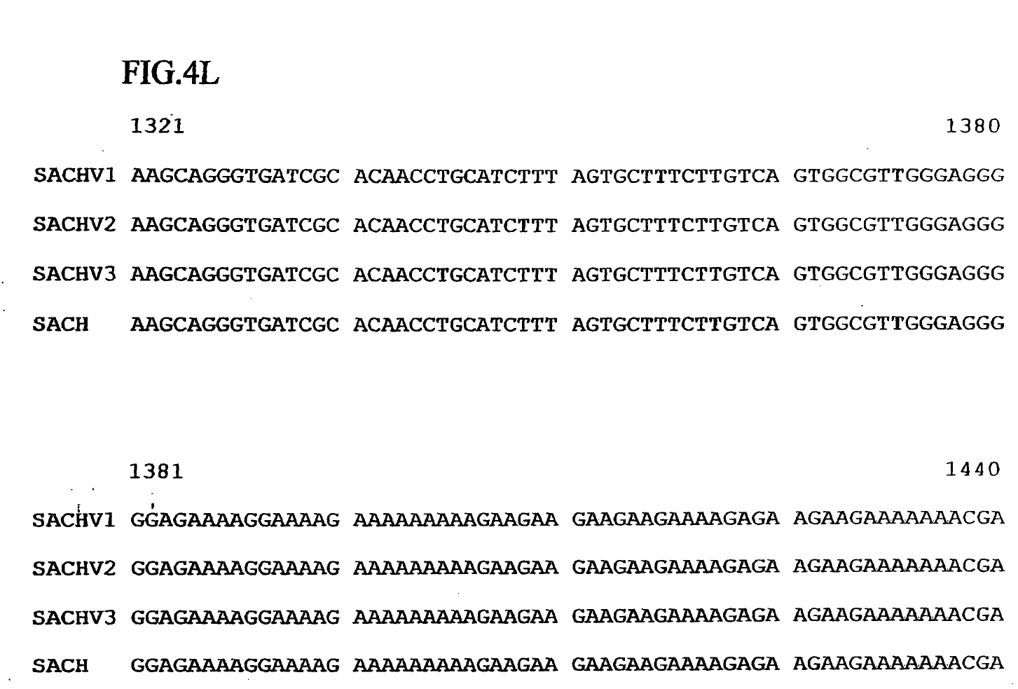

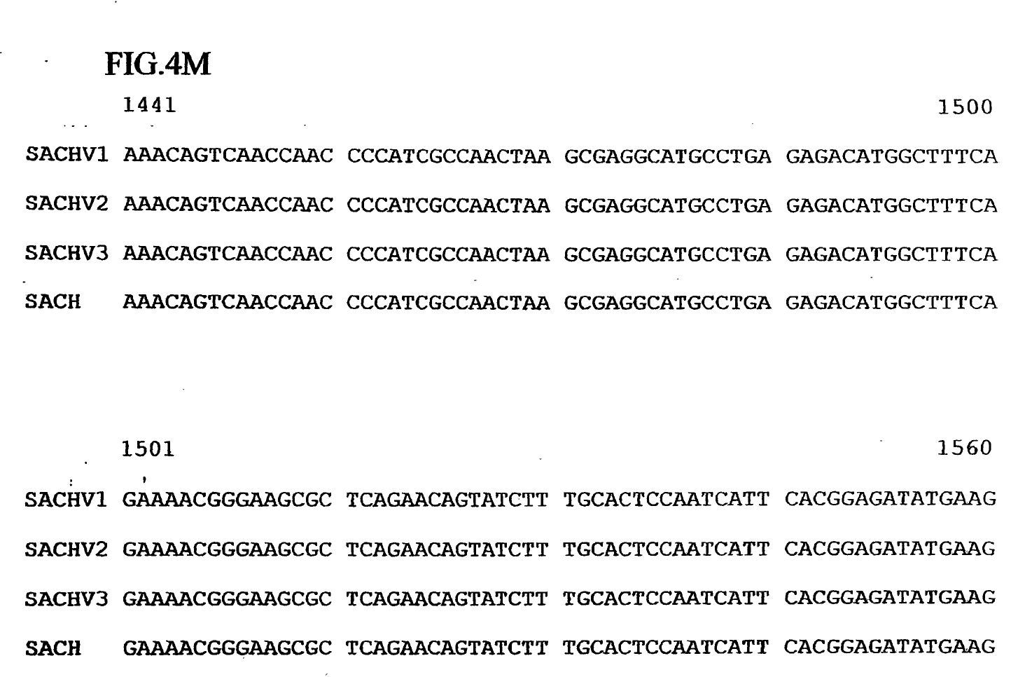

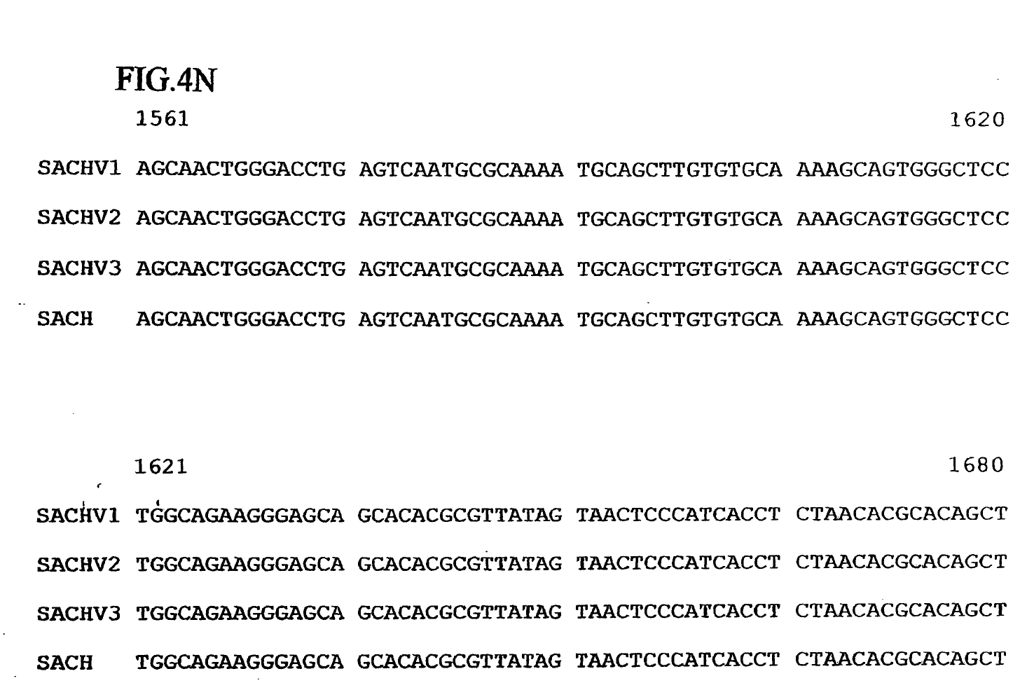

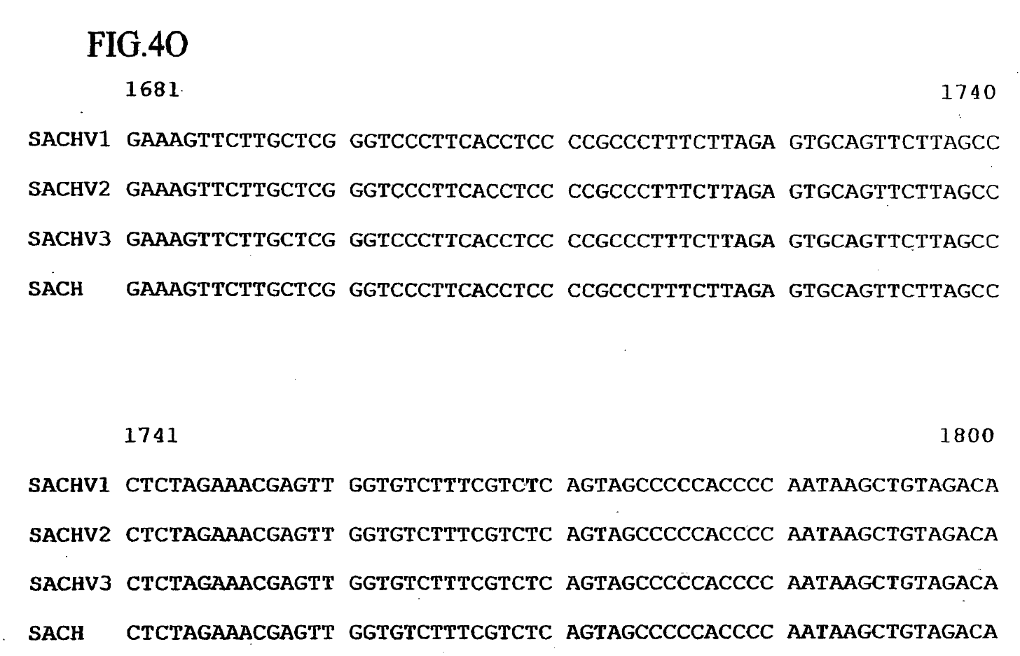

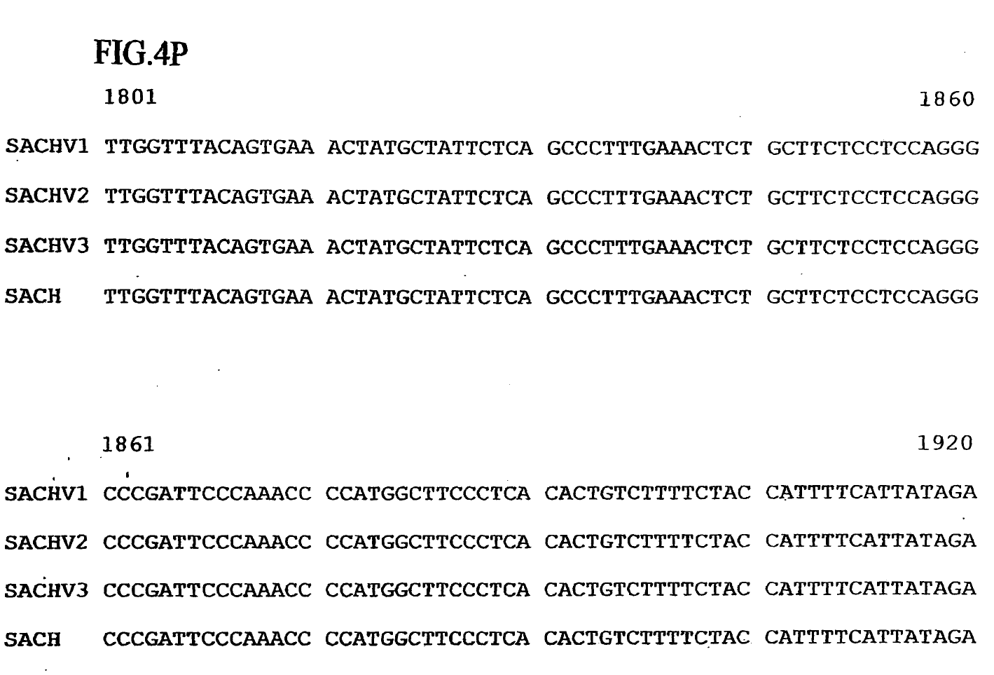

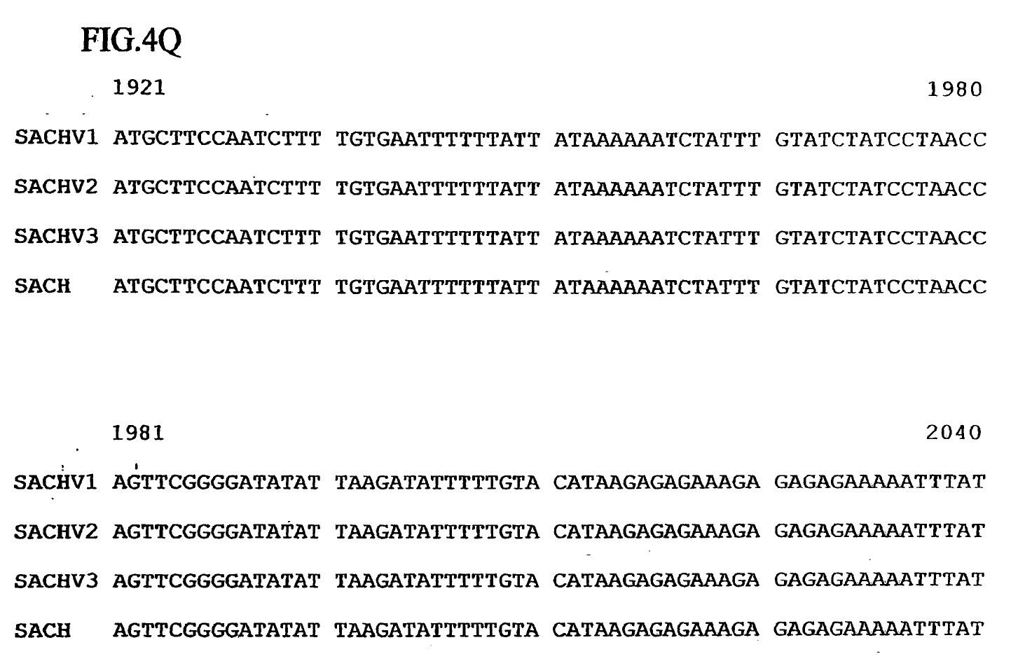

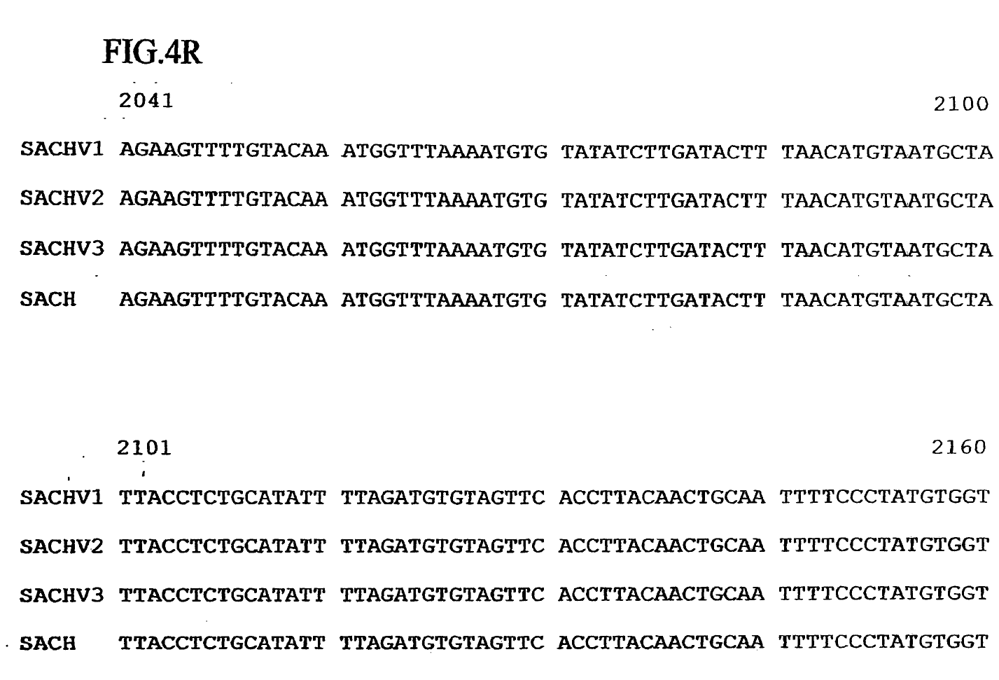

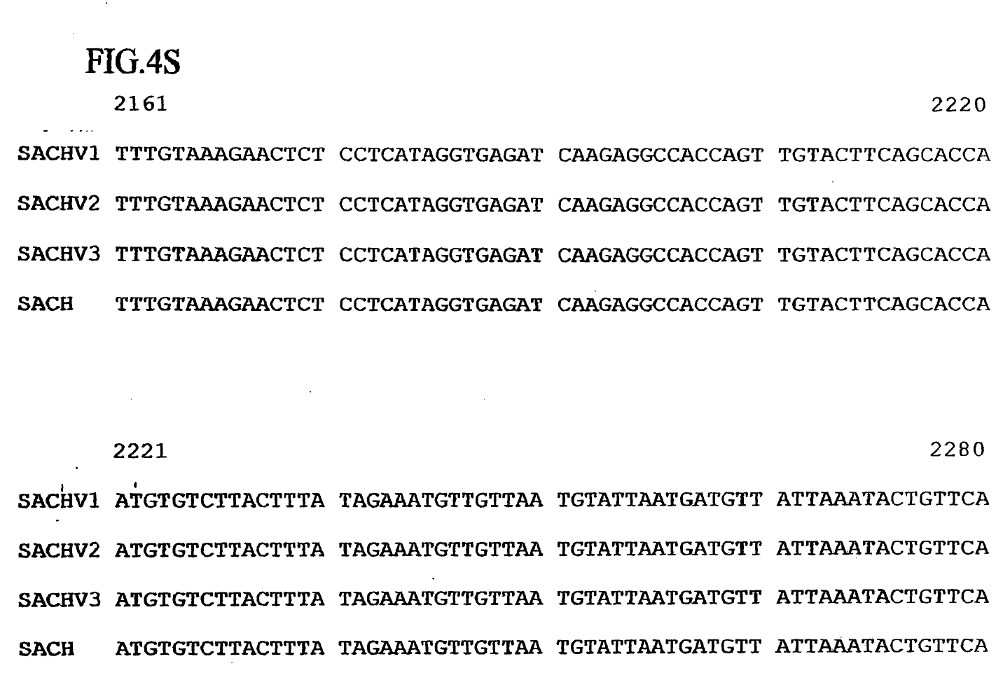

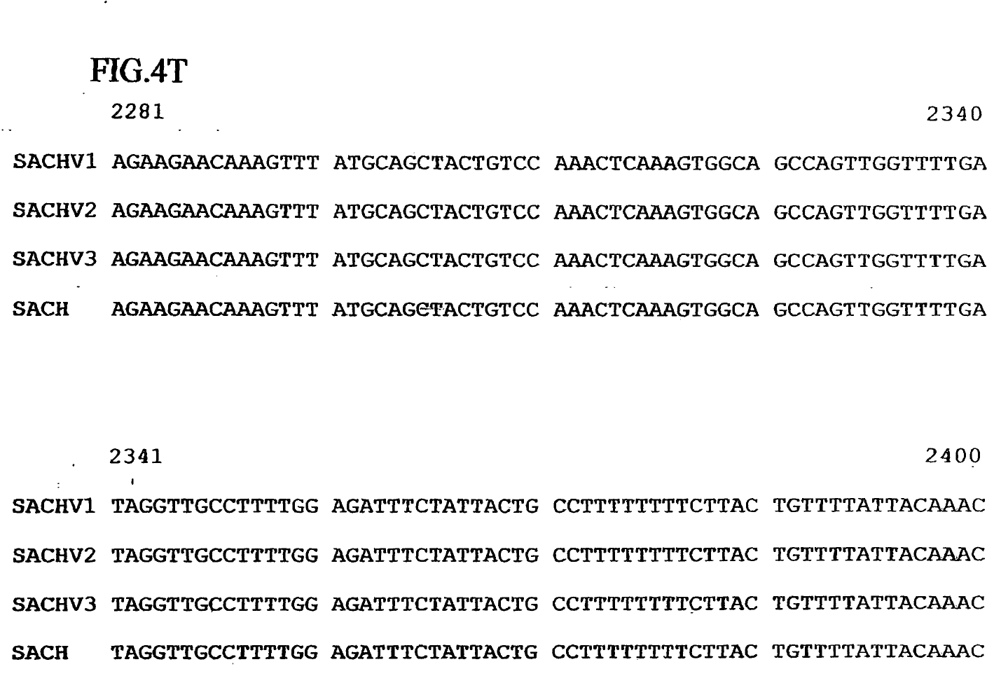

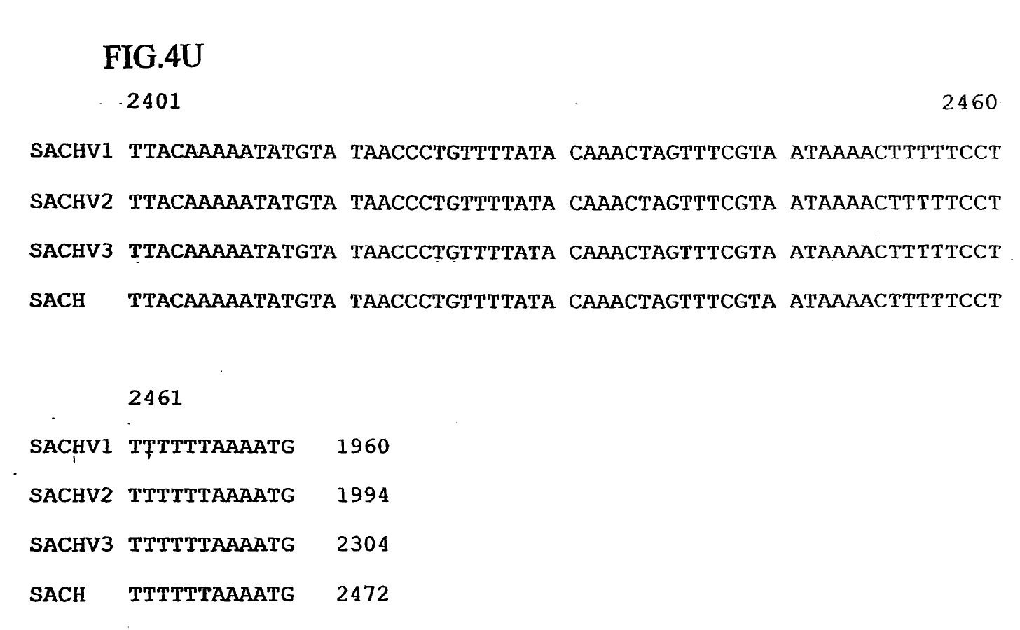

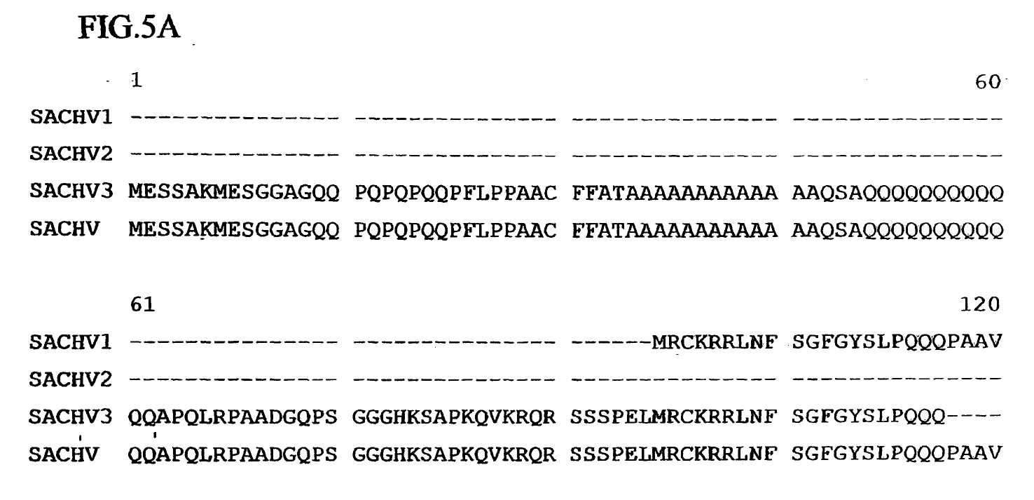

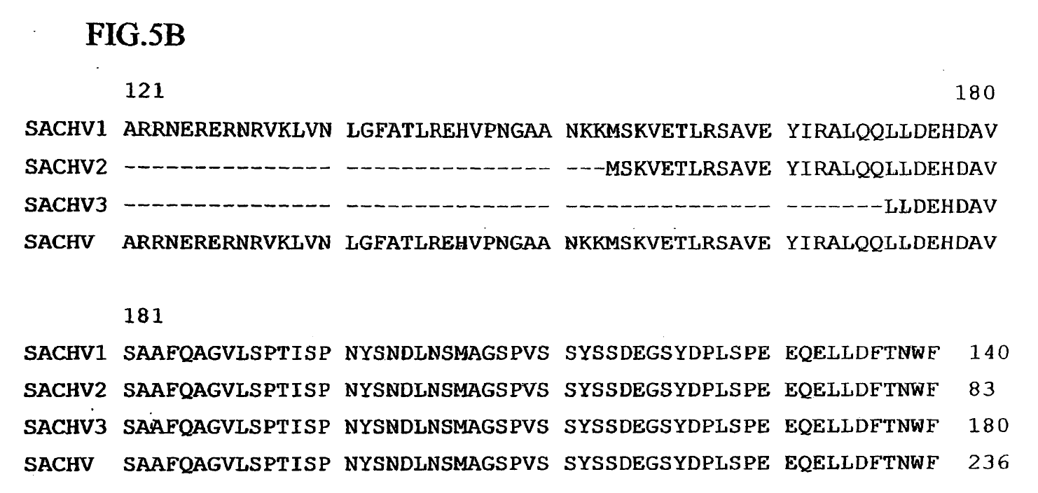

The full-length of the SACHV1 cDNA is a 1960 bp clone containing a 420 bp open reading frame (ORF) extending from nucleotides 345 to 764, which corresponds to an encoded protein of 140 amino acid residues with a predicted molecular mass of 15.7 kDa. The full-length of the SACHV2 cDNA is a 1994 bp clone containing a 249 bp ORF extending from nucleotides 550 to 798, which corresponds to an encoded protein of 83 amino acid residues with a predicted molecular mass of 9.1% Da. The full-length of the SACHV3 cDNA is a 2304 bp clone containing a 540 bp ORF extending from nucleotides 569 to 1108, which corresponds to an encoded protein of 180 amino acid residues with a predicted molecular mass of 19.1 kDa. To determine the variations (insertion/deletion) in sequences of SACHV1, SACHV2 and SACHV3 cDNA clones, an alignment of SACH nucleotide/amino acid sequence with these clones was performed (FIGS. 4 and 5). The results indicate that three genetic deletions were found in the aligned sequences. This information demonstrates that SACHV1 is a 512 bp deletion in the sequence of SACH from nucleotides 199 to 710; SACHV2 is a 478 bp deletion in the sequence of SACH from nucleotides 489 to 966; and SACHV3 is a 168 bp deletion in the sequence of SACH from nucleotides 918 to 1085.

In the invention, a search of ESTs deposited in dbEST (Boguski et al., (1993) Nat Genet. 4: 332-3) at NCBI was performed. Three ESTs were found to confirm the missing region described in SACHV1, SACHV2 and SACHV3. One EST (GenBank accession number BE791033), which confirmed the absence of 512 bp region in SACHV1 nucleotide sequence, was found to have been isolated from a SCLC cDNA library. This suggests that the absence of the 512 bp nucleotide fragment located between nucleotides 198 and 199 of SACHV1 may be a useful marker for SCLC diagnosis. One EST (GenBank accession number B1117134), which confirmed the absence of 478 bp region on SACHV2 nucleotide sequences, was found to have been isolated from a SCLC cDNA library. This suggests that the absence of the 478 bp nucleotide fragment located between nucleotides 488 and 489 of SACHV2 may be a useful marker for SCLC diagnosis. One EST (GenBank accession number BE502909), which confirmed the absence of 168 bp region on SACHV3 nucleotide sequences, was found to have been isolated from a lung carcinoid cDNA library. This suggests that the absence of 168 bp nucleotide fragment located between nucleotides 917 and 918 of SACHV3 is an important marker in association with lung carcinoid.

Therefore, any nucleotide fragments comprising nucleotides 196-201 of SACHV1, nucleotides 486-491 of SACHV2 or nucleotides 915-920 of SACHV3 may be used as probes for determining the presence of the variants under highly stringent conditions. An alternative approach is that any set of primers for amplifying the fragments containing nucleotides 196-201 of SACHV1, nucleotides 486-491 of SACHV2 or nucleotides 915-920 of SACHV3 may be used for determining the presence of the variants.

According to the present invention, the polypeptides encoded by human SACH-related gene variants (SACHV1, SACHV2 and SACHV3) and fragments thereof may be produced through genetic engineering techniques. In this case, they are produced by appropriate host cells that have been transformed by DNAs that code the polypeptides or fragments thereof. The nucleotide sequence encoding the polypeptide of the human SACH-related gene variants or fragment thereof is inserted into an appropriate expression vector, i.e., a vector which contains the necessary elements for the transcription and translation of the inserted coding sequence in a suitable host. The nucleic acid sequence is inserted into the vector in a manner that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing the sequences encoding the polypeptides of the human SACH-related gene variants and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express the polypeptide-coding sequence. These include, but are not limited to, microorganisms such as bacteria transformed with recombinant bacteriophage, plasmid, or cosmid DNA expression vector; yeast transformed with a yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and most preferably, the bacterium is E. coli.

Alternatively, the polypeptides encoded by human SACH-related gene variants or fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 431A peptide synthesizer (Perkin-Elmer).

According to the present invention, the fragments of the polypeptides and nucleic acid sequences of the human SACH-related gene variants are used as immunogens and primers or probes, respectively. It is preferable to use the purified fragments of the human SACH-related gene variants. The fragments may be produced by enzyme digestion, chemical cleavage of isolated or purified polypeptide or nucleic acid sequences, or chemical synthesis, and then may be isolated or purified. Such isolated or purified fragments of the polypeptides and nucleic acid sequences can be used directly as immunogens and primers or probes, respectively.

The present invention further provides the antibodies which specifically bind one or more out-surface epitopes of the polypeptides encoded by human SACH-related gene variants.

According to the present invention, immunization of mammals with immunogens described herein, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. No. 4,271,145 and U.S. Pat. No. 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci USA 93: 12394-9; Liu et al., (1997) Nat Genet 16:328-9; Siffert et al., (1998) Nat Genet 18: 45 to 8; Lukas et al., (2001) Cancer Res 61: 3212 to 9). Based on the cDNA libraries of the matched ESTs, SACHV1 and SACHV2 can be specifically associated with SCLC, whereas SACHV3 can be associated with lung carcinoid. Thus, the expression level of SACHV1, SACHV2 and SACHV3, each relative to SACH, may be a useful indicator for screening of patients suspected of having cancers, or more specifically, the SCLC or lung carcinoid. This suggests that the index of relative expression level (mRNA or protein) may be associated with an increased susceptibility to cancers or SCLC, more preferably, lung carcinoid. Fragments of SACHV1, SACHV2 and SACHV3 transcripts (mRNAs) may be detected by RT-PCR approach. Polypeptides encoded by the SACH-related gene variants may be determined by the binding of antibodies to these polypeptides. These approaches may be performed in accordance with conventional methods well known by persons skilled in the art.

The subject invention also provides methods for diagnosing the diseases associated with the deficiency of human SACH gene in a mammal, in particular, lung cancer, e.g., SCLC and lung carcinoid.

The method for diagnosing the diseases associated with the deficiency of human SACH genes may be performed by detecting the nucleotide sequences of SACHV1, SACHV2 or SACHV3 of the invention, which comprises the steps of: (1) extracting total RNA of cells obtained from a mammal; (2) amplifying the RNA by reverse transcriptase-polymerase chain reaction (RT-PCR) with a set of primers to obtain a cDNA comprising the fragments comprising nucleotides 196-201 of SEQ ID NO: 1 or nucleotides 486-491 of SEQ ID NO: 3 or nucleotides 915-920 of SEQ ID NO: 5; and (3) detecting whether the cDNA sample is obtained. If necessary, the amount of the obtained cDNA sample may be detected.

In this embodiment, a forward primer may be designed to have a sequence comprising nucleotides 196-201 of SEQ ID NO: 1 and a reverse primer may be designed to have a sequence complementary to the nucleotides of SEQ ID NO: 1 at any other locations downstream of nucleotide 201; or a forward primer has a sequence comprising nucleotides 486-491 of SEQ ID NO: 3 and a reverse primer has a sequence complementary to the nucleotides of SEQ ID NO: 3 at any other locations downstream of nucleotide 491; or a forward primer has a sequence comprising nucleotides 915-920 of SEQ ID NO: 5 and a reverse primer has a sequence complementary to the nucleotides of SEQ ID NO: 5 at any other locations downstream of nucleotide 920. Alternatively, the reverse primer may be designed to have a sequence complementary to the nucleotides of SEQ ID NO: 1 containing nucleotides 196-201 and the forward primer may be designed to have a sequence comprising the nucleotides of SEQ ID NO: 1 at any other locations upstream of nucleotide 196; or the reverse primer has a sequence complementary to the nucleotides of SEQ ID NO: 3 containing nucleotides 486-491 and the forward primer has a sequence comprising the nucleotides of SEQ ID NO: 3 at any other locations upstream of nucleotide 486; or the reverse primer has a sequence complementary to the nucleotides of SEQ ID NO: 5 containing nucleotides 915-920 and the forward primer has a sequence comprising the nucleotides of SEQ ID NO: 5 at any other locations upstream of nucleotide 915. In this case, only SACHV1, SACHV2 and SACHV3 will be amplified.

Alternatively, the forward primer may be designed to have a sequence comprising the nucleotides of SEQ ID NO: 1 at any locations upstream of nucleotide 196 and the reverse primer may be designed to have a sequence complementary to the nucleotides of SEQ ID NO: 1 at any other locations downstream of nucleotide 201; or the forward primer has a sequence comprising the nucleotides of SEQ ID NO: 3 at any locations upstream of nucleotide 486 and the reverse primer has a sequence complementary to the nucleotides of SEQ ID NO: 3 at any other locations downstream of nucleotide 491; or the forward primer has a sequence comprising the nucleotides of SEQ ID NO: 5 at any locations upstream of nucleotide 915 and the reverse primer has a sequence complementary to the nucleotides of SEQ ID NO: 5 at any other locations downstream of nucleotide 920. In this case, SACHV1, SACHV2 or SACHV3, together with SACH in a sample, will be amplified. The length of the PCR fragment from SACHV1 will be 512 bp shorter than that from SACH; the length of the PCR fragment from SACHV2 will be 478 bp shorter than that from SACH; the length of the PCR fragment from SACHV3 will be 168 bp shorter than that from SACH.

Preferably, the primers of the invention contain 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of gene variants (SACHV1, SACHV2 and SACHV3) can be used. PCR fragments are analyzed on a 1% agarose gel using five microliters (10%) of the amplified products. The intensity of the signals may be determined by using the Molecular Analyst program (version 1.4.1; Bio-Rad). Thus, the index of relative expression levels for each co-amplified PCR products may be calculated based on the intensity of signals.

The RT-PCR experiment may be performed according to the manufacturer's instructions (Boehringer Mannheim). A 50 μl reaction mixture containing 211 total RNA (0.1 μg/μl), 1 μl each primer (20 μM), 1 μl each dNTP (10 mM), 2.5 μl DTT solution (100 mM), 10 μl 5×RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of human SACH gene is performed by detecting the nucleotide sequence of SACHV1, SACHV2 or SACHV3, which comprises the steps of: (1) extracting the total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid selected from the group consisting of SEQ ID NOs: 1, 3 and 5, and the fragments thereof; and (4) detecting whether the cDNA sample hybridizes with the nucleic acid of SEQ ID NO: 1, 3 or 5, or the fragments thereof. If necessary, the amount of hybridized sample may be detected.

The expression of gene variants can be analyzed using the Northern Blot hybridization approach. Specific fragments comprising nucleotides 196-201 of the SACHV1, nucleotides 486-491 of the SACHV2 or nucleotides 915-920 of the SACHV3 may be amplified by polymerase chain reaction (PCR) using a primer set designed for RT-PCR. The amplified PCR fragment may be labeled and serve as a probe to hybridize the membranes containing the total RNAs extracted from the samples under the conditions of 55° C. in a suitable hybridization solution for 3 hours. The blots may be washed twice in 2×SSC, 0.1% SDS at room temperature for 15 minutes each, followed by two washes in 0.1×SSC and 0.1% SDS at 65° C. for 20 minutes each. After these washes, the blots may be rinsed briefly in suitable washing buffer and incubated in a blocking solution for 30 minutes, and then incubated in a suitable antibody solution for 30 minutes. The blots may be washed in a washing buffer for 30 minutes, and equilibrated in a suitable detection buffer before detecting the signals. Alternatively, the presence of gene variants (cDNAs or PCR) can be detected using a microarray approach. The cDNAs or PCR products corresponding to the nucleotide sequences of the present invention may be immobilized on a suitable substrate such as a glass slide. Hybridization can be performed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency of human SACH gene may also be performed by detecting the polypeptides encoded by SACHV1, SACHV2 and SACHV3 of the invention. For instance, the polypeptides in protein samples obtained from the mammal may be determined by, but is not limited to, the immunoassay wherein the antibody specifically binding to the polypeptides of the invention is contacted with the protein sample, and the antibody-polypeptide complex is detected. If necessary, the amount of the antibody-polypeptide complexes can be determined.

The polypeptides encoded by the gene variants can be expressed in prokaryotic cells by using suitable prokaryotic expression vectors. The cDNA fragments of SACHV1, SACHV2 or SACHV3 genes encoding the amino acid coding sequence may be PCR amplified with restriction enzyme digestion sites incorporated in the 5′ and 3′ ends, respectively. For example, specific fragments which comprise nucleotides 902-925 (encoding amino acid residues 112-119) of the SACHV3 may be PCR amplified. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 hours. The bacterially-expressed proteins may be purified.

The polypeptides encoded by SACH-related gene variants can be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of each of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectamine Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins/polypeptides. After these proteins/polypeptides are purified, monoclonal antibodies against these purified proteins/polypeptides (SACHV1, SACHV2 and SACHV3) may be generated using hybridoma technique according to the conventional methods (de StGroth and Scheidegger, (1980) J Immunol Methods 35:1-21; Cote et al. (1983) Proc Natl Acad Sci USA 80: 2026-30; and Kozbor et al. (1985) J Immunol Methods 81:31-42).

According to the present invention, the presence of the polypeptides encoded by the gene variants in samples of lung cancers may be determined by, but is not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 hour at 100 V (e.g., Bio-Rad). The proteins can be immunoblotted with specific antibodies. For example, membrane blotted with extracted proteins may be blocked with suitable buffers such as a 3% solution of BSA or 3% solution of nonfat milk powder in TBST buffer (10 mM Tris-HCl, pH 8.0, 150 mM NaCl, 0.1% Tween 20) and incubated with monoclonal antibody directed against the polypeptides encoded by the gene variants. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human clones from a SCLC and a lung carcinoid cDNA libraries were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at the National Center for Biotechnology Information (NCBI) with a significance cutoff of p<10⁻¹⁰. ESTs representing putative SACH encoding gene were identified during the course of EST generation.

### Isolation of cDNA Clones

Three cDNA clones exhibiting EST sequences similar to the SACH gene were isolated from the cDNA libraries and named SACHV1, SACHV2 and SACHV3. The inserts of these clones were subsequently excised in vivo from the λZAP Express vector using the ExAssist/XLOLR helper phage system (Stratagene). Phagemid particles were excised by coinfecting XL1-BLUE MRF⁺ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution Analysis

The coding sequence for each cDNA clone was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variants (insertions or deletions) with a significance cutoff of p<10⁻¹⁰.

### REFERENCES

Altschul et al., Gapped BLAST and PSI-BLAST: a new generation of protein database search programs, Nucleic Acids Res, 25: 3389-3402, (1997).
Ausubel et al., Current protocols in Molecular Biology, John Wiley & Sons, New York N.Y., ch. 9, 13, and 16, (1995).
Ball et al., Identification of a human achaete-scute homolog highly expressed in neuroendocrine tumors. Proc Natl Acad Sci USA. 90:5648-52, (1993).
Bhattacharjee et al., Classification of human lung carcinomas by mRNA expression profiling reveals distinct adenocarcinoma subclasses. Proc Natl Acad Sci USA. 98:13790-5, (2001).
Boguski et al., dbEST—database for “expressed sequence tags”. Nat Genet. 4: 332-3, (1993).
Carney, D. N. The biology of lung cancer. Curr. Opin. Oncol. 4: 292-8, (1992a).
Carney, D. N. Biology of small-cell lung cancer. Lancet 339: 843-6, (1992b).
Cote et al., Generation of human monoclonal antibodies reactive with cellular antigens, Proc Natl Acad Sci USA 80: 2026-30 (1983).
de StGroth and Scheidegger, Production of monoclonal antibodies: strategy and tactics, J Immunol Methods 35:1-21, (1980).
Gish and States, Identification of protein coding regions by database similarity search, Nat Genet, 3:266-272, (1993).
Ihde and Minna, Non-small cell lung cancer. Part II: Treatment. Curr. Probl. Cancer 15: 105-54, (1991).
Kozbor et al., Specific immunoglobulin production and enhanced tumorigenicity following ascites growth of human hybridomas, J Immunol Methods, 81:31-42 (1985).
Liu et al., Silent mutation induces exon skipping of fibrillin-1 gene in Marfan syndrome. Nat Genet 16:328-9, (1997).
Lukas et al., Alternative and aberrant messenger RNA splicing of the mdm2 oncogene in invasive breast cancer. Cancer Res 61:3212-9, (2001).
Massari and Murre, Helix-loop-helix proteins: regulators of transcription in eucaryotic organisms. Mol Cell Biol. 20:429-40 (2000).
Roberge et al., A strategy for a convergent synthesis of N-linked glycopeptides on a solid support. Science 269:202-4, (1995).
Sambrook, J. Cold Spring Harbor Press, Plainview N.Y., ch. 4, 8, and 16-17.
Sethi, Science, medicine, and the future. Lung cancer, BMJ, 314: 652-655, (1997)
Siffert et al., Association of a human G-protein beta3 subunit variant with hypertension. Nat Genet, 18:45-8, (1998).
Smyth et al., The impact of chemotherapy on small cell carcinoma of the bronchus. Q J Med, 61: 969-76, (1986).
Sriuranpong et al., Notch signaling induces cell cycle arrest in small cell lung cancer cells. Cancer Res. 61:3200-5, (2001).
Stallings-Mann et al., Alternative splicing of exon 3 of the human growth hormone receptor is the result of an unusual genetic polymorphism. Proc Natl Acad Sci USA 93:12394-9, (1996).
Strausberg, R. EST Accession No. BE791033, B1117134, BE502909.
Strausberg, R. nucleotide Accession No. BC001638
Sugita et al., Combined use of oligonucleotide and tissue microarrays identifies cancer/testis antigens as biomarkers in lung carcinoma. Cancer Res. 62:3971-9, (2002).

1. An isolated polypeptide selected from the group consisting of SEQ ID NOs: 2, 4, and 6, and fragments comprising the amino acid residues 112 to 119 of SEQ ID NO: 6.

2. An isolated nucleic acid comprising a nucleotide sequence selected from the group consisting of SEQ ID NOs: 1, 3 and 5, and fragments thereof.

3. The isolated nucleic acid of claim 2, wherein the fragments comprise nucleotides 196 to 201 of SEQ ID NO: 1.

4. The isolated nucleic acid of claim 2, wherein the fragments comprise nucleotides 486 to 491 of SEQ ID NO: 3.

5. The isolated nucleic acid of claim 2, wherein the fragments comprise nucleotides 915 to 920 of SEQ ID NO: 5.

6. An expression vector comprising the nucleic acid of claim 2.

7. A host cell transformed with the expression vector of claim 6.

8. A method for producing a polypeptide, which comprises the steps of:
(1) culturing the host cell of claim 7 under a condition suitable for the expression of the polypeptide; and
(2) recovering the polypeptide from the host cell culture.

9. An antibody specifically binding to the polypeptide of claim 1.

10. A method for diagnosing a disease associated with the deficiency of a SACH gene in a mammal, which comprises detecting the nucleic acid of claim 2 or a polypeptide encoded thereby.

11. The method of claim 10, wherein the detection of the nucleic acid comprises the steps of:
(1) extracting the total RNA from a sample obtained from the mammal;
(2) amplifying the RNA by reverse transcriptase-polymerase chain reaction (RT-PCR) to obtain a cDNA sample;
(3) bringing the cDNA sample into contact with the nucleic acid; and
(4) detecting whether the cDNA hybridizes with the nucleic acid.

12. The method of claim 11 further comprising the step of determining the amount of the hybridized sample.

13. The method of claim 10, wherein the detection of the nucleic acid comprises the steps of:
(1) extracting the total RNAs of cells obtained from the mammal;
(2) amplifying the RNA by reverse transcriptase-polymerase chain reaction (RT-PCR) with a set of primers to obtain a cDNA comprising the fragments comprising nucleotides 196 to 201 of SEQ ID NO: 1 or nucleotides 486 to 491 of SEQ ID NO: 3 or nucleotides 915 to 920 of SEQ ID NO: 5; and
(3) detecting whether the cDNA is obtained.

14. The method of claim 13, wherein the forward primer has a sequence comprising the nucleotides 196 to 201 of SEQ ID NO: 1 and the reverse primer has a sequence complementary to the nucleotides of SEQ ID NO: 1 at any other locations downstream of nucleotide 201, or alternatively, the reverse primer has a sequence complementary to the nucleotides of SEQ ID NO: 1 containing nucleotides 196 to 201 and the forward primer has a sequence comprising the nucleotides of SEQ ID NO: 1 at any other locations upstream of nucleotide 196.

15. The method of claim 13, wherein the forward primer has a sequence comprising the nucleotides 486 to 491 of SEQ ID NO: 3 and the reverse primer has a sequence complementary to the sequence complementary to the nucleotides of SEQ ID NO: 3 at any other locations downstream of nucleotide 491, or alternatively, the reverse primer has a sequence complementary to the nucleotides of SEQ ID NO: 3 containing nucleotides 486 to 491 and the forward primer has a sequence comprising the nucleotides of SEQ ID NO: 3 at any other locations upstream of nucleotide 486.

16. The method of claim 13, wherein the forward primer has a sequence comprising the nucleotides between 915 to 920 of SEQ ID NO: 5 and the reverse primer has a sequence complementary to the nucleotides of SEQ ID NO: 5 at any other locations downstream of nucleotide 920, or alternatively, the reverse primer has a sequence complementary to the nucleotides of SEQ ID NO: 5 containing nucleotides between 915 to 920 and the forward primer has a sequence comprising the nucleotides of SEQ ID NO: 5 at any other locations upstream of nucleotide 915.

17. The method of claim 13, wherein the forward primer has a sequence comprising the nucleotides of SEQ ID NO: 1 at any other locations upstream of nucleotide 196 and the reverse primer has a sequence complementary to the nucleotides of SEQ ID NO: 1 at any other locations downstream of nucleotide 201.

18. The method of claim 13, wherein the forward primer has a sequence comprising the nucleotides of SEQ ID NO: 3 at any other locations upstream of nucleotide 486 and the reverse primer has a sequence complementary to the nucleotides of SEQ ID NO: 3 at any other locations downstream of nucleotide 491.

19. The method of claim 13, wherein the forward primer has a sequence the nucleotides of SEQ ID NO: 5 at any other locations upstream of nucleotide 915 and the reverse primer has a sequence complementary to the nucleotides of SEQ ID NO: 5 at any other locations downstream of nucleotide 920.

20. The method of claim 17, wherein the cDNA sample amplified from SEQ ID NO: 1 is 512 bp shorter than that from SACH.

21. The method of claim 18, wherein the cDNA sample amplified from SEQ ID NO: 3 is 478 bp shorter than that from SACH.

22. The method of claim 19, wherein the cDNA sample amplified from SEQ ID NO: 5 is 168 bp shorter than that from SACH.

23. The method of claim 13 further comprising the step of detecting the amount of the amplified cDNA sample.

24. The method of claim 10, wherein the detection of the polypeptide comprises the steps of contacting an antibody that specifically binds to the polypeptide with protein samples extracted from the mammal, and detecting whether an antibody-polypeptide complex is formed.

25. The method of claim 24 further comprising the step of determining the amount of the antibody-polypeptide complex.


SEQUENCE LISTING (6 sequences)

SEQ ID NO: 1 - DNA, 1960 bp, ARTIFICIAL SEQUENCE
  A Variant of a human SACH gene
ggcacgaggcttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctccccgccctttcttagagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgccttttttttcttactgttttattacaaacttacaaaaatatgtataaccctgttttatacaaactagtttcgtaataaaactttttcctttttttaaaatg

SEQ ID NO: 2 - protein, 139 aa, ARTIFICIAL SEQUENCE
  A peptide encoded by a variant of a human SACH gene
MRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNW

SEQ ID NO: 3 - DNA, 1994 bp, ARTIFICIAL SEQUENCE
  A variant of a human SACH gene
ggcacgaggcttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctccccgccctttcttagagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgccttttttttcttactgttttattacaaacttacaaaaatatgtataaccctgttttatacaaactagtttcgtaataaaactttttcctttttttaaaatg

SEQ ID NO: 4 - protein, 82 aa, ARTIFICIAL SEQUENCE
  A peptide encoded by a variant of a human SACH gene
MSKVETLRSAVEYIRALQQLLDEHDAVSAAFQAGVLSPTISPNYSNDLNSMAGSPVSSYSSDEGSYDPLSPEEQELLDFTNW

SEQ ID NO: 5 - DNA, 2304 bp, ARTIFICIAL SEQUENCE
  A variant of a human SACH gene
ggcacgaggcttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctccccgccctttcttagagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgccttttttttcttactgttttattacaaacttacaaaaatatgtataaccctgttttatacaaactagtttcgtaataaaactttttcctttttttaaaatg

SEQ ID NO: 6 - protein, 79 aa, ARTIFICIAL SEQUENCE
  A peptide encoded by a variant of a human SACH gene
MESSAKMESGGAGQQPQPQPQQPFLPPAACFFATAAAAAAAAAAAAAQSAQQQQQQQQQQQQAPQLRPAADGQPSGGGH